(19) United States
(12) Patent Application Publication
(10) Pub. No.: US 2007/0006343 A1
(43) Pub. Date: Jan. 4, 2007

(54) Modified oleosins

(75) Inventors: Maurice M. Moloney, Calgary (CA); Rodrigo Marins Siloto, Calgary (CA)

Correspondence Address: BERESKIN AND PARR, 40 KING STREET WEST, BOX 401, TORONTO, ON, M5H 3Y2, CA

(73) Assignee: SEMBIOSYS GENETICS INC, Calgary (CA)

(21) Appl. No.: 10/561,178

(22) PCT Filed: Jun. 18, 2004
(86) PCT No.: PCT/CA04/00904

§ 371 Date: Jun. 9, 2006

Related U.S. Application Data
(60) Provisional application No. 60/479,851, filed on Jun. 20, 2003.

(51) Int. Cl. A01H 5/00; C07H 21/04; C12N 9/10; C12N 15/82; C12N 5/04

(52) U.S. Cl. 800/281; 435/193; 435/419; 435/468; 536/23.2

(57) ABSTRACT

The present invention describes novel polypeptide structures based on oleosin molecules which are capable of being targeted to oil bodies in plants. The modified oleosin polypeptides are obtained by performing modifications in the hydrophobic domain encoding sequence of an oleosin cDNA. The present invention describes methods to obtain such polypeptides in vivo. The novel oleosins may be used to deliver a recombinant (non-oleosin) protein to oil bodies.

### FIELD OF THE INVENTION

The present invention relates to modified oleosin polypeptides which contain sufficient information to be targeted to oil bodies in plants. The modified polypeptides are obtained by performing modifications in the hydrophobic domain encoding sequence of an oleosin cDNA. The present invention describes methods to obtain such polypeptides in vivo. The modified oleosins may be used to deliver a heterologous protein to oil bodies.

### BACKGROUND OF THE INVENTION

Oil seed plants represent an economically important renewable ressource for the production of oils, proteins and other valuable products. In such oil seed plants, neutral lipids (typically triglycerides) are stored within the seed in subcellular organelles termed oil bodies which serve as a source of energy to the germinating seedling. Electron microscopy studies have demonstrated that these organelles are synthesized on the endoplasmic reticulum (ER) membrane and surrounded by a single layer of phospholipid as a half-unit membrane. This layer is coated by proteins that are specifically targeted to oil bodies, known as oil body proteins. The most abundant class of oil body proteins present in oil bodies, are called oleosins.

All known oleosins share unique features in primary sequence and domain distribution. Sequence alignments show that these proteins can be divided into three distinct regions. The first 50 to 70 residues at the N-terminal portion, a central lipophilic region known as hydrophobic domain with about 65 to 75 residues and the last 50 to 90 residues at the C-terminal portion (Huang, 1992, Annu. Rev. Plant. Physiol. Plant Mol. Biol. 43: 177-200). According to protease protection assays the hydrophobic domain of the oleosin is completely protected by the oil body while the N- and C-terminal regions face the cytoplasm (Huang, 1992, Ann Rev Plant Physiol Plant Mol Biol 43: 177-200; Abell et al, 1997, Plant Cell 9: 1481-1493).

The hydrophobic domain is also the most conserved region when oleosins from different species are compared. A comprehensive analysis of this domain reveals the presence of a 12-residue motif containing three prolines, a serine and an alanine completely conserved in all sequenced oleosins. This motif, known as proline knot motif (PKM), is probably responsible for flexing the hydrophobic domain 180° in the middle, forming two anti-parallel hydrophobic chains. Tzen et al. (1992), JBC 267(22): 15626-15634 have proposed a model of a maize oleosin attached to an oil body. This model suggests that the anti-parallel chains interact with each other through hydrogen bonding between the side chains of serines, threonines.

The prior art has disclosed the ability to use oleosins as carriers for proteins and peptides and the subsequent use of oil bodies containing these protein fusions as purification vehicles for recombinant proteins and peptides (U.S. Pat. No. 5,650,554). The prior art also discloses a number of modifications to oleosins which are either permissible or not permissible in order to ensure efficient targeting of oleosins or oleosin-protein fusions to oil bodies. Van Rooijen and Moloney (1995a) Plant Physiol 109: 1353-1361 showed that the loss of the cytoplasmically exposed C-terminus of oleosin proteins did not significantly impair targeting nor oleosin (or oleosin-fusion) stability. On the other hand, removal of the cytoplasmically exposed N-terminus of oleosin significantly impaired the efficiency of targeting to oil bodies of oleosin-protein fusions (ibid). Abell et al (1997) Plant Cell 9:1481-1493, demonstrated the importance of the proline knot motif as an essential component for targeting to the oil body itself, but determined the proline knot motif was not required for the incorporation of an oleosin polypeptide into the ER membrane. To date, to the knowledge of the inventors, in only one instance the prior art teaches oleosin modifications within the hydrophobic domain and outside of the proline knot. Abell et al, (2002) JBC 277:8602-8610. substituted the hydrophobic regions flanking the protein knot, called H(N) and H(C), respectively to produce modified oleosins of the formulae: N-terminal-H(N)-PKM-H(N)-C-terminal or N-terminal-H(C)-PKM-H(C)-C-terminal. These modified oleosins were shown to associate correctly to the ER suggesting that the native primary sequence was not a prerequisite for co-translation on the ER. However, the authors did not test whether these polypeptides would target correctly to oil bodies if expressed in transgenic plants. Given the requirement for the proline knot motif for oil body targeting, but not for ER association, it cannot be concluded that such a variant could target correctly to oil bodies expressed in seeds of transgenic plants. In addition, the prior art does not provide any information with respect to the effect on oil body targeting of oleosin variants in which the length of the hydrophobic core is modified. Thus the prior art provides only a limited amount of oleosin variants having a modified hydrophobic core. Moreover, with respect to those variants available in the prior art, targeting and the ability to serve as carriers for recombinant proteins is unknown.

There is a need in the art for oleosin variants. Such variants will have utility as protein anchors, which could be designed for greater or lesser affinity of oleosin-protein fusions to oil bodies. Protein anchors of higher or lower affinity than native oleosins are useful in the recovery of recombinant proteins from oil bodies.

### SUMMARY OF THE INVENTION

The present invention describes modified oleosin polypeptides which are capable of targeting to oil bodies in vivo. The modified oleosins are also capable of directing the targeting of a heterologous polypeptide covalently fused to the modified oleosin to the oil body.

In particular, the present invention discloses a series of oleosin-derived proteins in which the length or composition of the hydrophobic domains have been altered, but which successfully target to oil bodies in vivo and which are capable of directing the association of a recombinant protein with the oil bodies. Furthermore, the present invention discloses the procedures for the creation of transgenic plants capable of expressing the novel oil body-targeted proteins and protein-fusions. Methods for the recovery, isolation and purification of the modified oil bodies and the proteins associated with the oil body surface, are also disclosed. The present invention provides modified oleosin molecules capable of targeting to an oil body wherein the modified oleosin comprises a proline knot motif and (i) a hydrophobic domain reduced in size by the removal of at least one amino acid residue on both the amino-terminal end and the carboxy-terminal end of the hydrophobic domain or (ii) a hydrophobic domain increased in size by the addition of at least one amino acid residue on both the amino terminal and carboxy terminal end of the hydrophobic domain. In preferred embodiments the oleosin molecules comprise an oleosin molecule from which an equal number of amino acid residues is removed or added to the hydrophobic domain, for example the hydrophobic domain of the oleosin may be reduced in size by the removal of for example between 2 and 28 amino acid residues on both the amino-terminal and the carboxy-terminal end of the hydrophobic domain or by the addition of for example between 2 and 28 amino acid residues to both the amino-terminal and carboxy-terminal end of the hydrophobic domain.

Further disclosed are seeds and oil bodies derived from these plants.

The inventors further disclose the uses of modified oleosin protein fusions on oil bodies for easy recovery of the recombinant protein linked to the modified oleosin.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 depicts the nucleotide sequence (SEQ ID NO. 1) and deduced amino acid sequence (SEQ ID NO. 2) of the full length oleosin clone (Oleo-FL). The predicted amino acid sequence is shown in single letter code. The deduced amino acid sequence of the N-terminal domain is in bold, N-terminal hydrophobic region (Chain HN) is in italics, the deduced amino acid sequence of the proline knot motif is underlined, the deduced amino acid sequence of the C-terminal hydrophobic region (Chain HC) sequence is in italics and bold and finally the C-terminal domain is in italics and underlined.

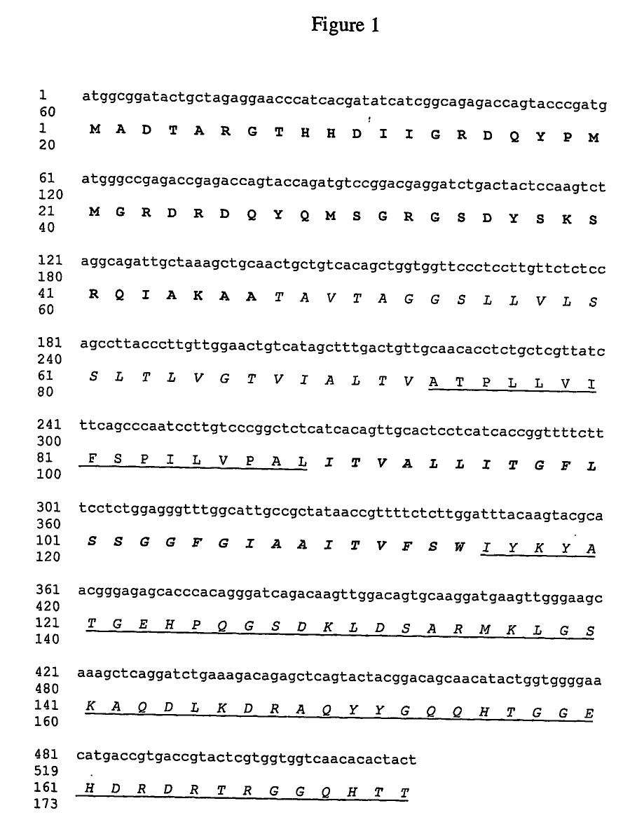

FIG. 2 depicts the nucleotide sequence (SEQ ID NO. 3) and deduced amino acid sequence (SEQ ID NO. 4) of the OleoH23P clone. The predicted amino acid sequence is shown in single letter code. The deduced amino acid sequence of the N-terminal domain is in bold, the modified N-terminal hydrophobic region (Chain HN) is in italics, the deduced amino acid sequence of the proline knot motif is underlined, the deduced amino acid sequence of the modified C-terminal hydrophobic region (Chain HC) sequence is in italics and bold and finally the C-terminal domain is in italics and underlined.

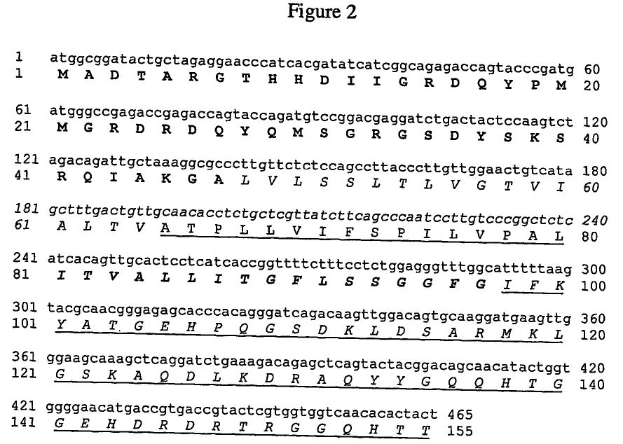

FIG. 3 depicts the nucleotide sequence (SEQ ID NO. 5) and deduced amino acid sequence (SEQ ID NO. 6) of the OleoH3P clone. The predicted amino acid sequence is shown in single letter code. The deduced amino acid sequence of the N-terminal domain is in bold, the modified N-terminal hydrophobic region (Chain HN) is in italics, the deduced amino acid sequence of the proline knot motif is underlined, the deduced amino acid sequence of the modified C-terminal hydrophobic region (Chain HC) sequence is in italics and bold and finally the C-terminal domain is in italics and underlined.

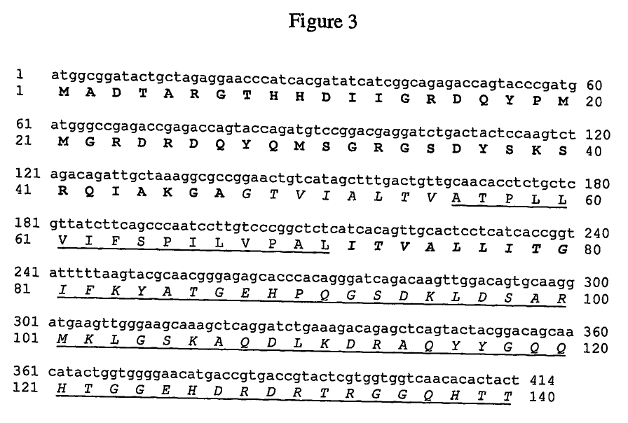

FIG. 4 depicts the nucleotide sequence (SEQ ID NO. 7) and deduced amino acid sequence (SEQ ID NO. 8) of the OleoP clone. The predicted amino acid sequence is shown in single letter code. The deduced amino acid sequence of the N-terminal domain is in bold, the modified N-terminal hydrophobic region (Chain HN) is in italics, the deduced amino acid sequence of the proline knot motif is underlined, the deduced amino acid sequence of the modified C-terminal hydrophobic region (Chain HC) sequence is in italics and bold and finally the C-terminal domain is in italics and underlined.

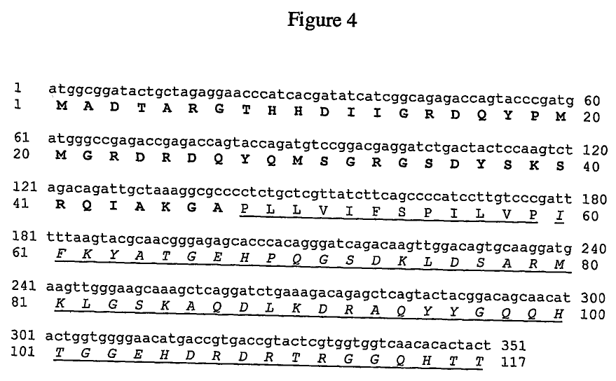

FIG. 5 depicts the nucleotide sequence (SEQ ID NO. 9) and deduced amino acid sequence (SEQ ID NO. 10) of the OleoH12P clone. The predicted amino acid sequence is shown in single letter code. The deduced amino acid sequence of the N-terminal domain is in bold, the modified N-terminal hydrophobic region (Chain HN) is in italics, the deduced amino acid sequence of the proline knot motif is underlined, the deduced amino acid sequence of the modified C-terminal hydrophobic region (Chain HC) sequence is in italics and bold and finally the C-terminal domain is in italics and underlined.

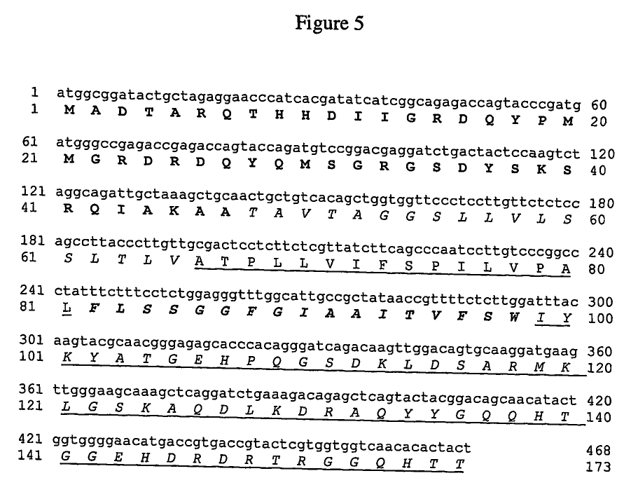

FIG. 6 depicts the nucleotide sequence (SEQ ID NO. 11) and deduced amino acid sequence (SEQ ID NO. 12) of the OleoH1P clone. The predicted amino acid sequence is shown in single letter code. The deduced amino acid sequence of the N-terminal domain is in bold, the modified N-terminal hydrophobic region (Chain HN) is in italics, the deduced amino acid sequence of the proline knot motif is underlined, the deduced amino acid sequence of the modified C-terminal hydrophobic region (Chain HC) sequence is in italics and bold and finally the C terminal domain is in italics and underlined.

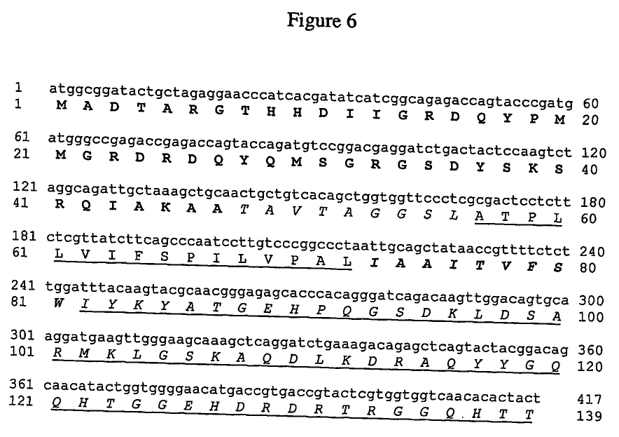

FIG. 7 depicts the nucleotide sequence (SEQ ID NO. 13) and deduced amino acid sequence (SEQ ID NO. 14) of the Oleo-double clone. The predicted amino acid sequence is shown in single letter code. The deduced amino acid sequence of the N-terminal domain is in bold, the modified N-terminal hydrophobic region (Chain HN) is in italics, the extra amino acid residues are bold and underlined, the deduced amino acid sequence of the proline knot motif is underlined, the deduced amino acid sequence of the modified C-terminal hydrophobic region (Chain HC) sequence is in italics and bold and finally the C-terminal domain is in italics and underlined.

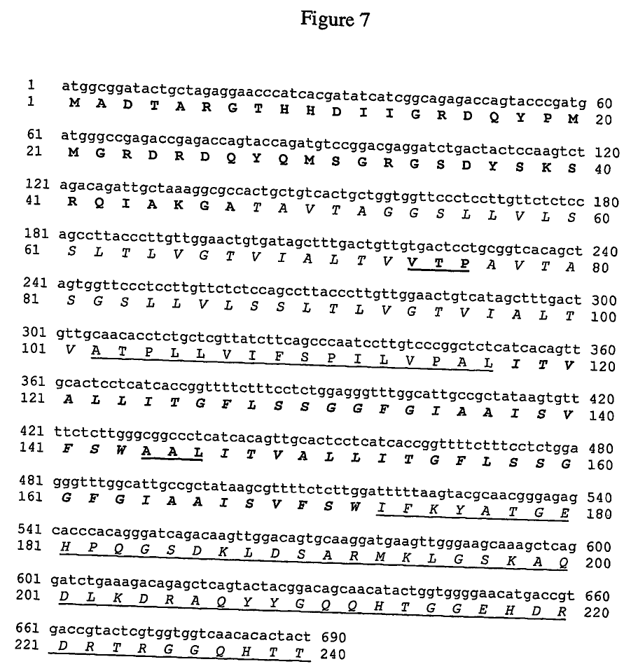

FIG. 8: Amino acid sequence of 18 kDa oleosin from Arabidopsis thaliana (Oleo-FL) and summary of the modifications performed. (a) Primary structure of Oleo-FL. (b) Description of each domain in Oleo-FL according to the proposed model for oleosin structure. (c) General description of modifications performed in the hydrophobic chains.

FIG. 9: Scheme for construction of DNA sequence encoding for oleosins with reduced hydrophobic domain stretch following “Direction1” mode. (a) Scheme of wild-type Oleo-FL_domain structure. (b) Amplification of the N-terminal sequence of oleosin and amplification of reduced versions of hydrophobic domain from oleosin. c) Fusion of N-terminal fragment to different versions of hydrophobic domain. Amplification of the C-terminal sequence of oleosin. (d) Fusion of C-terminal fragment to N-terminal/hydrophobic domains creating Oleo H23P, OleoH3P and OleoP.

FIG. 10: Scheme for construction of DNA sequence encoding for oleosins with reduced hydrophobic domain stretch following “Direction2” mode a) Scheme of wild-type Oleo-FL domain structure. (b) Amplification of the N-terminal sequence plus amplification of reduced versions of hydrophobic chain HN of oleosin (c) Fusion of N-terminal reduced hydrophobic domains to proline knot motif. (d) Fusion of N- and C-terminal domains creating Oleo H12P and OleoH1P.

FIG. 11: Scheme for construction of DNA sequence encoding for oleosins with increased hydrophobic domain stretch. a) Scheme of Oleo-FL_domain structure. (b) Amplification of the N-terminal sequence of oleosin and amplification of Hydrophobic chain HN. (c) Ligation of N-terminal sequence of oleosin and hydrophobic chain HN. Amplification of hydrophobic domain of oleosin. (d) Ligation to create fragment FrNTNextCD. Amplification of FrNTNext CD and hydrophobic chain HC. (e) Ligation of FrNTNextCD and FrHCext are ligated creating the fragment FrNTNextCDCext. Amplification of FrNTNextCDCext. (f) Ligation of FrNTNextCDCext and FrCT to create Oleodouble fragment and subsequent amplification.

FIG. 12: Construct scheme of the binary vector carrying a modified oleosin fused to GFP. (a) The oleosin sequence is inserted in the plasmid pGFP in the sites XhoI and SpeI (pOLG) in frame with GFP. (b) The fusion is excised with NcoI and PstI and inserted in the plasmid pUBIp+is3′ (pUBOLG) between ubiquitin promoter and terminator. (c) The cassette is sub-cloned into pBluescript KS (+) in the EcoRI site (pKsUBOLG). (d) The cassette is inserted in the binary vector pSBS3000 in the BamHI and KpnI sites forming the binary vector carrying a modified oleosin fused to GFP (e).

FIG. 13: Modifications in Oleo-FL hydrophobic domain. (a) Structure of the normal Oleo-FL hydrophobic domain showing the proline knot motif and the anti-parallel hydrophobic chains. (b) Modified versions of Oleo-FL hydrophobic domain containing reduced and increased hydrophobic chains.

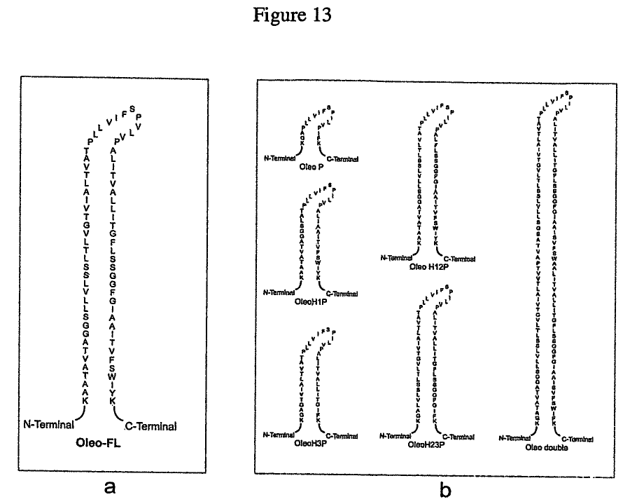

FIG. 14: (A) Accumulation of modified oleosins in seeds from transgenic Arabidopsis lines. Total seed protein extracts from seeds of about twenty-five independent transformants can be analyzed using western blot with anti-GFP antibody. GFP standards are used in each experiment and the membranes can be scanned in a densitometer. The amounts of each band can be normalized with the GFP standards and plotted. (B) Average accumulation of recombinant modified oleosin fused to GFP (nanograms of recombinant oleosin per microgram of total seed protein).

FIG. 15: Subcellular localization of modified oleosins fused to GFP examined in confocal microscopy. The polypeptides are expressed in Arabidopsis seeds, showing association in vivo with oil bodies. Embryos from mature seeds expressing modified oleosins are isolated and examined with a Zeiss LSM 510 laser scanning confocal microscope. Green fluorescence corresponds to GFP. (A) Oleo-FL, (B) Oleo-H12P, (C)Oleo-H23P, (D) Oleo-H1P, (E) Oleo-H3P, (F) Oleo-P, (G) Oleo-Double

FIG. 16: Differential affinity of full-length oleosin (Oleo-FL) and modified oleosins (Oleo-H12P, Oleo-H23P, Oleo-H1P, Oleo-H3P, Oleo-P and Oleo-double) for oil bodies as demonstrated by washing with different detergent solutions.

### DETAILED DESCRIPTION OF THE INVENTION

I. Definitions

Unless defined otherwise, all technical and scientific terms used herein shall have the same meaning as is commonly understood by one skilled in the art to which the present invention belongs. Where permitted, all patents, applications, published applications, and other publications, including nucleic acid and polypeptide sequences from GenBank, SwissPro and other databases referred to in the disclosure are incorporated by reference in their entirety.

By “At least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tₘ, which in sodium containing buffers is a function of the sodium ion concentration and temperature (Tₘ=81.5° C.−16.6 (Log₁₀ [Na⁺])+0.41(%(G+C)−600/l), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tₘ, for example if nucleic acid molecules are sought that have a>95% identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5× sodium chloride/sodium citrate (SSC)/5× Denhardt's solution/1.0% SDS at Tₘ −5° C. based on the above equation, followed by a wash of 0.2×SSC/0.1% SDS at 60° C. Moderately stringent hybridization conditions include a washing step in 3×SSC at 42° C. It is understood however that equivalent stringencies may be achieved using alternative buffers, salts and temperatures: Additional guidance regarding hybridization conditions may be found in: Current Protocols in Molecular Biology, John Wiley & Sons, N.Y., 1989, 6.3.1.-6.3.6 and in: Sambrook et al., Molecular Cloning, a Laboratory Manual, Cold Spring Harbor Laboratory Press, 1989, Vol. 3.

The term “chimeric” as used herein in the context of nucleic acid sequences refers to at least two linked nucleic acid sequences which are not naturally linked. Chimeric nucleic acid sequences include linked nucleic acid sequences of different natural origins. For example a nucleic acid sequence constituting a plant promoter linked to a nucleic acid sequence encoding human insulin is considered chimeric. Chimeric nucleic acid sequences also may comprise nucleic acid sequences of the same natural origin, provided they are not naturally linked. For example a nucleic acid sequence constituting a promoter obtained from a particular cell-type may be linked to a nucleic acid sequence encoding a polypeptide obtained from that same cell-type, but not normally linked to the nucleic acid sequence constituting the promoter. Chimeric nucleic acid sequences also include nucleic acid sequences comprising any naturally occurring nucleic acid sequence linked to any non-naturally occurring nucleic acid sequence.

The phrase “conservative amino acid substitution”, as used herein, is one in which one amino acid residue is replaced with another amino acid residue without abolishing the protein's desired properties. Typically, conservative substitutions involve amino acids with similar charge, hydrophobicity and/or overall size. Examples of conservative amino acid substitutions include replacement of the amino acid glutamate with the amino acid aspartate, alanine with valine, leucine or isoleucine.

The phrase “functional variant” as used herein means a protein or nucleic acid sequence that differs from a specified sequence although the protein or nucleic acid sequence retains the same function as the specified sequence. Functional variants include amino acid sequences that differ from a specified sequence due to conservative amino acid substitutions. Functional variants include nucleic acid sequences that can hybridize to a complement of a specified sequence under at least moderately stringent hybridization conditions. Functional variants of both amino acid and nucleic acid sequences include sequences that are substantially identical as defined herein. Functional variants of modified oleosins include modified oleosins that permit the formation of oil bodies in vivo and oleosins that permit the targeting in vivo of heterologous proteins linked to oleosins to oil bodies.

As used herein the terms “modified oleosin” and “modified oleosin polypeptide”, which may be used interchangeable herein, refer to any and all modified oleosin polypeptides that comprise (a) a proline knot motif and (b) a hydrophobic domain wherein the hydrophobic domain is modified by the removal or addition of at least one amino acid residue on both the amino terminal (HN) and carboxy terminal (HC) chains of the hydrophobic domain. Modified oleosin polypeptides include the polypeptides listed in SEQ ID NOS:4, 6, 8, 10, 12 or 14 as well as a polypeptide molecule comprising a sequence of amino acid residues which is (i) substantially identical to the amino acid sequences constituting the modified oleosin polypeptides set forth herein or (ii) encoded by a nucleic acid sequence capable of hybridizing under at least moderately stringent conditions to any nucleic acid sequence encoding the modified oleosin set forth herein or capable of hybridizing under at least moderately stringent conditions to any nucleic acid sequence encoding modified oleosin set forth herein but for the use of synonymous codons.

The term “nucleic acid sequence” as used herein refers to a sequence of nucleoside or nucleotide monomers consisting of naturally occurring bases, sugars and intersugar (backbone) linkages. The term also includes modified or substituted sequences comprising non-naturally occurring monomers or portions thereof. The nucleic acid sequences of the present invention may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine.

The terms “nucleic acid sequence encoding modified oleosin” and “nucleic acid sequence encoding a modified oleosin polypeptide”, which may be used interchangeably herein, refer to any and all nucleic acid sequences encoding a modified oleosin polypeptide as defined herein, including the oleosin polypeptides listed in SEQ ID NOS:4, 6, 8, 10, 12 or 14. Nucleic acid sequences encoding a modified oleosin polypeptide further include any and all nucleic acid sequences which (i) encode polypeptides that are substantially identical to the modified oleosin polypeptide sequences set forth herein; or (ii) hybridize to any nucleic acid sequences set forth herein under at least moderately stringent hybridization conditions or which would hybridize thereto under at least moderately stringent conditions but for the use of synonymous codons.

The term “oil body” or “oil bodies” as used herein refers to any oil or fat storage organelle in a cell, including any plant cell (described in for example: Huang (1992) Ann. Rev. Plant Mol. Biol. 43: 177-200).

The term “oleosin” as used herein means an oil body protein found in plants that comprises three domains: 1) an N-terminal domain; 2) a centrally located hydrophobic domain; and 3) a C-terminal domain. Nucleic acid sequences encoding oleosins are known to the art. These include for example the Arabidopsis oleosin (Van Rooijen et al (1991) Plant Mol. Bio. 18:1177-1179); the maize oleosin (Qu and Huang (1990) J. Biol. Chem. Vol. 265 4:2238-2243); rapeseed oleosin (Lee and Huang (1991) Plant Physiol. 96:1395-1397); and the carrot oleosin (Hatzopoulos et al (1990) Plant Cell Vol. 2, 457-467.)

The “proline knot motif” is a motif within the hydrophobic domain of an oleosin composed of three proline residues distributed over a twelve to fifteen residue region. The prolines are predicted to mediate a turn, which would facilitate the formation of an antiparallel α-helix or α-strand (Huang (1992) Annu. Rev. Plant Physiol. Plant Mol. Biol. 43: 177-200). The proline knot motif is flanked by the HN and HC regions within the hydrophobic domain.

By the term “substantially identical” it is meant that two polypeptide sequences preferably are at least 75% identical, and more preferably are at least 85% identical and most preferably at least 95% identical, for example 96%, 97%, 98% or 99% identical. In order to determine the percentage of identity between two polypeptide sequences the amino acid sequences of such two sequences are aligned, preferably using the Clustal W algorithm (Thompson, J D, Higgins D G, Gibson T J, 1994, Nucleic Acids Res. 22 (22): 4673-4680, together with BLOSUM 62 scoring matrix (Henikoff S. and Henikoff J. G., 1992, Proc. Natl. Acad. Sci. USA 89: 10915-10919) and a gap opening penalty of 10 and gap extension penalty of 0.1, so that the highest order match is obtained between two sequences wherein at least 50% of the total length of one of the sequences is involved in the alignment. Other methods that may be used to align sequences are the alignment method of Needleman and Wunsch (J. Mol. Biol., 1970, 48: 443), as revised by Smith and Waterman (Adv. Appl. Math., 1981, 2: 482) so that the highest order match is obtained between the two sequences and the number of identical amino acids is determined between the two sequences. Other methods to calculate the percentage identity between two amino acid sequences are generally art recognized and include, for example, those described by Carillo and Lipton (SIAM J. Applied Math., 1988, 48:1073) and those described in Computational Molecular Biology, Lesk, e.d. Oxford University Press, New York, 1988, Biocomputing: Informatics and Genomics Projects. Generally, computer programs will be employed for such calculations. Computer programs that may be used in this regard include, but are not limited to, GCG (Devereux et al., Nucleic Acids Res., 1984, 12: 387) BLASTP, BLASTN and FASTA (Altschul et al., J. Molec. Biol., 1990: 215: 403).

II. Modified Oleosins

The present invention relates to a series of oleosin-derived proteins in which the length or composition of the hydrophobic domains have been altered, but which successfully target to oil bodies in vivo and which are capable of directing the association of a recombinant protein with the oil bodies. In particular, the present invention provides a modified oleosin polypeptide comprising (a) a proline knot motif and (b) a hydrophobic domain wherein the hydrophobic domain is modified by the removal or addition of at least one amino acid residue on both the amino terminal (HN) and carboxy terminal (HC) chains of the hydrophobic domain. In one embodiment, the present invention provides a modified oleosin polypeptide capable of targeting to an oil body wherein the modified oleosin comprises a proline knot motif and a hydrophobic domain reduced in size by the removal of at least one amino acid residue on both the amino-terminal end and the carboxy-terminal end of the hydrophobic domain. In another embodiment, the present invention provides a modified oleosin polypeptide capable of targeting to an oil body wherein the modified oleosin comprises a proline knot motif and a hydrophobic domain increased in size by the addition of at least one amino acid residue on both the amino terminal and carboxy terminal end of the hydrophobic domain.

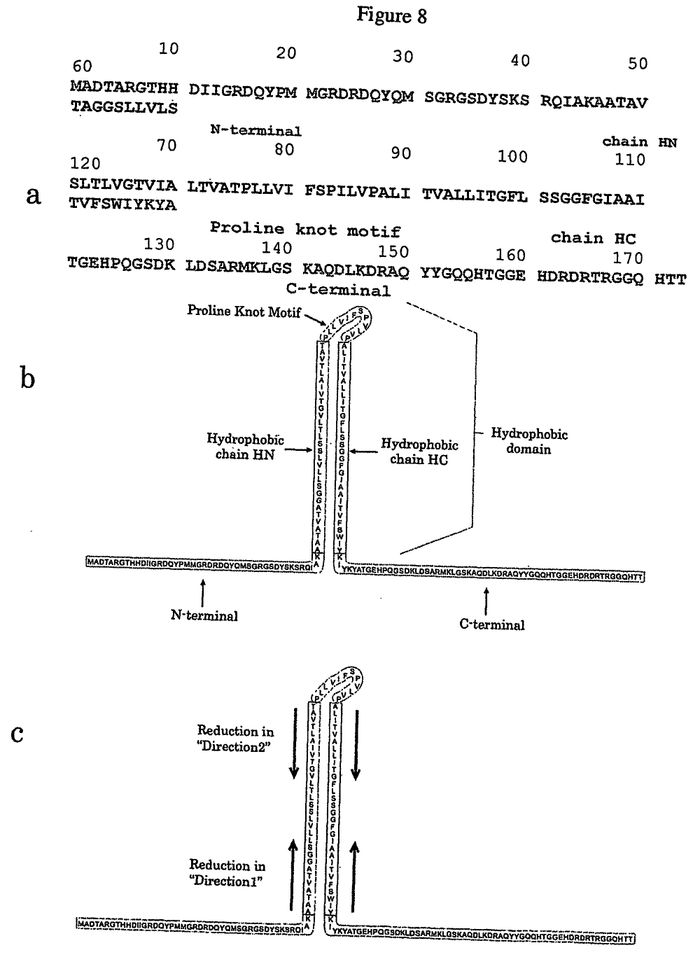

As previously mentioned, the amino acid sequence of oleosin comprises 3 domains, 1) the N-terminal domain; 2) the hydrophobic domain; and 3) the C-terminal domain. The hydrophobic domain contains the hydrophobic chain HN, the proline knot motif and the hydrophobic chain HC. (This is shown schematically in FIG. 8a.) In accordance with the present invention, modifications are performed in the hydrophobic chains sequences that flank the proline knot motif. Specifically, the present invention describes methods to reduce and increase the length of the hydrophobic chains. When modifying the HN and HC chains, the length of the HN chain should remain similar to the length of the HC chain in order to preserve predicted oleosin structure (Tzen, et al. 1992, JBC 267(22): 15626-15634).

In preferred embodiments, the modified polypeptides comprise an oleosin polypeptide from which an equal number of amino acid residues is removed or added to the hydrophobic domain, for example the hydrophobic domain of the oleosin may be reduced in size by the removal of for example between 2 and 28 amino acid residues on both the amino-terminal and the carboxy-terminal end of the hydrophobic domain or by the addition of for example between 2 and 28 amino acid residues to both the amino-terminal and carboxy-terminal end of the hydrophobic domain.

The hydrophobic chain HN can be further divided into 3 sub-domains 1a, 2a and 3a and the hydrophobic chain HC can be further divided into 3 sub-domains, 1b, 2b, and 3b. Each of the sub-domains of the hydrophobic chains of these sub-domains contain approximately the same number of hydrophobic residues. In a preferred embodiment, one or more of the sub-domains is removed from both the hydrophobic chain HN and the hydrophobic chain HC. In a further preferred embodiment, a modified oleosin is created wherein individual sub-domains are removed. For example both sub-domain 1a and 1b are removed, both sub-domain 2a and 2b are removed or both sub-domain 3a and 3b are removed. In a further preferred embodiment, a modified oleosin is created wherein multiple sub-domains are removed. For example sub-domain 1a, 1b, 2a and 2b; 2a, 2b, 3a and 3b; 1a, 1b, 3a and 3b; or 1a, 1b, 2a, 2b, 3a and 3b are removed.

The present invention can be used to modify any oleosin of interest, including any plant oleosin such for example an Arabidopsis thaliana oleosin, a Brassica oleosin, or a corn oleosin. In a preferred embodiment, the oleosin is substantially identical to a plant oleosin such as the oleosin isolated from Arabidopsis thaliana (SEQ ID NO: 2 or 15) or Brassica napus (SEQ ID NO:16). In a preferred embodiment, the oleosin used to prepare the modified oleosin is substantially identical to the sequence shown in SEQ ID NO:2. In a specific embodiment the cDNA of Oleo-FL encoding the 18 kDa oleosin in Arabidopsis thaliana is used to prepare modified oleosins (SEQ ID NO:1).

The nucleotide sequences of the domains from the Arabidopsis thaliana oleosin are as follows: The N-terminal domain consists of nucleotides 1-141 (SEQ ID NO:17) from the Oleo-FL clone or is modified as shown in SEQ ID NO:18; The hydrophobic chain HN domain 1a is represented by nucleotides 142-168 (SEQ ID NO:19) from the Oleo-FL clone or is modified as shown in SEQ ID NO:20; The hydrophobic chain HN domain 2a is represented by nucleotides 169-195 (SEQ ID NO:21) from the Oleo-FL clone; The hydrophobic chain HN domain 3a is represented by nucleotides 196-219 (SEQ ID NO:22) from the Oleo-FL clone; The proline knot motif is defined as nucleotides 225-261 (SEQ ID NO:23) or nucleotides 220-267 (SEQ ID NO:24) of the Oleo-FL clone; The hydrophobic chain HC domain 1b is represented by nucleotides 319-345 (SEQ ID NO:25) from the Oleo-FL clone or is modified as shown in SEQ ID NO:26; The hydrophobic chain HC domain 2b is represented by nucleotides 295-318 (SEQ ID NO:27) from the Oleo-FL clone; The hydrophobic chain HN domain 3b is represented by nucleotides 268-294 (SEQ ID NO:28) from the Oleo-FL clone; The C-terminal domain consists of nucleotides 346-519 (SEQ ID NO:29) from the Oleo-FL clone or is modified as shown in SEQ ID NO:30.

The amino acid sequences of the domains from the Arabidopsis thaliana oleosin are as follows: The N-terminal domain consists of amino acids 1-47 (SEQ ID NO:31) from the Oleo-FL clone or is modified as shown in SEQ ID NO:32; The hydrophobic chain HN domain 1a is represented by amino acids 48-56 (SEQ ID NO:33) from the Oleo-FL clone or is modified as shown in SEQ ID NO:34; The hydrophobic chain HN domain 2a is represented by amino acids 57-65 (SEQ ID NO:35) from the Oleo-FL clone; The hydrophobic chain HN domain 3a is represented by amino acids 66-73 (SEQ ID NO:36) from the Oleo-FL clone; The proline knot motif is defined as amino acids 76-87 (SEQ ID NO:37) or amino acids 74-89 (SEQ ID NO:38) of the Oleo-FL clone; The hydrophobic chain HC domain 1b is represented by amino acids 107-115 (SEQ ID NO:39) from the Oleo-FL clone or is modified as shown in SEQ ID NO:40; The hydrophobic chain HC domain 2b is represented by amino acids 99-106 (SEQ ID NO:41) from the Oleo-FL clone; The hydrophobic chain HN domain 3b is represented by amino acids 88-98 (SEQ ID NO:42) from the Oleo-FL clone; The C-terminal domain consists of amino acids 116-173 (SEQ ID NO:43) from the Oleo-FL clone or is modified as shown in SEQ ID NO:44.

The modified oleosin polypeptides of the invention comprise the proline knot motif and modified HN and HC chains. The modified oleosins may additionally comprise a N-terminal domain and/or a C-terminal domain, or portions or functional variants thereof. The modified oleosin preferably comprises a N-terminal domain, or a functional variant thereof and optionally comprises a C-terminal domain, or a functional variant thereof. In a specific embodiment, the nucleotide sequence of the N-terminal domain is selected from the sequences SEQ ID NO:17 or SEQ ID NO:18 or portions or functional variants thereof. The nucleotide sequence of the proline knot motif is selected from the sequences SEQ ID NO:23 or SEQ ID NO:24 or functional variants thereof. The nucleotide sequence of the C-terminal domain is selected from the sequences SEQ ID NO:29 or SEQ ID NO:30 or portions or functional variants thereof. The modified oleosin polypeptides further comprises a modified hydrophobic chain HN and a hydrophobic chain HC. The hydrophobic chain HN is encoded by one or more of the following sequences: SEQ ID NO:19 (sub-domain 1a), SEQ ID NO:20 (modified sub-domain 1a), SEQ ID NO:21 (sub-domain 2a), SEQ ID NO:22 (sub-domain 3a) and the hydrophobic chain HC is encoded by one or more of the following sequences: SEQ ID NO:25 (sub-domain 1b), SEQ ID NO:26 (modified sub-domain 1b), SEQ ID NO:27 (sub-domain 2b) or SEQ ID NO:28 (sub-domain 3b).

In a specific embodiment in the modified oleosin polypeptides of the invention, the amino acid sequence of the N-terminal domain, if present, is selected from the sequences SEQ ID NO:31 or SEQ ID NO:32 or portions or functional variants thereof. The amino acid sequence of the proline knot motif is selected from the sequences SEQ ID NO:37 or SEQ ID NO:38 or functional variants thereof. The amino acid sequence of the C-terminal domain, if present, is selected from the sequences SEQ ID NO:43 or SEQ ID NO:44 or portions or functional variants thereof. The modified oleosin protein further comprises a modified hydrophobic chain HN and a hydrophobic chain HC. The hydrophobic chain HN consists of one or more of the following sequences: SEQ ID NO:33 (sub-domain 1a), SEQ ID NO:34 (modified sub-domain 1a), SEQ ID NO:35 (sub-domain 2a), SEQ ID NO:36 (sub-domain 3a) and the hydrophobic chain HC consists of one or more of the following sequences: SEQ ID NO:39 (sub-domain 1b), SEQ ID NO:40 (modified sub-domain 1b), SEQ ID NO:41 (sub-domain 2b) or SEQ ID NO:42 (sub-domain 3b).

(i) Reduction of Hydrophobic Domain Length

In a preferred embodiment, the length of the oleosin hydrophobic domain is reduced. Reduction of the size of the hydrophobic domain of oleosin may result in a novel emulsifier of reduced molecule weight, possibly more soluble in certain solvents than native oleosin. Shorter oleosin molecules are also expected to have reduced affinity to oil bodies and thus be more easily recovered from the oil body surface. Lower molecular weight modified oleosins might be expected to display more rapid diffusion that full length oleosins which would be useful to increase bioavailability of active molecules associated with the modified proteins.

In a preferred embodiment, the length of the hydrophobic domain length can be decreased by deleting regions coding for the hydrophobic chains HN and HC flanking the proline knot motif. Serial deletions in hydrophobic chains can be conducted in two different modes: deleting sequences from the N- and C-terminal ends to the proline knot motif (“Direction1”) or deleting sequences from the proline knot motif to the N- and C-terminal ends (“Direction2”) (FIG. 8c).

In a preferred embodiment, deletions in “Direction1” mode can be performed by obtaining reduced versions of the whole hydrophobic domain and subsequent fusion to N- and C-terminal domains. In a further preferred embodiment, equal amino acid residues are deleted from each of the hydrophobic chains HN and HC in each modification. In order to perform such modifications it is necessary to obtain three distinct fragments of oleosin DNA coding sequence: (a) N-terminal coding sequence, (b) reduced versions of hydrophobic domain (which includes the proline knot), (c) C-terminal coding sequence. In yet another preferred embodiment, the hydrophobic domains are reduced and the resulting modified oleosin sequences have the nucleotide sequences represented by Oleo-H23P, Oleo-H3P or Oleo-P which are SEQ ID NO:3, SEQ ID NO:5, or SEQ ID NO: 7 respectively and the amino acid sequences represented by Oleo-H23P, Oleo-H3P or Oleo-P which are SEQ ID NO:4, SEQ ID NO:6, or SEQ ID NO: 8 respectively. The invention further includes functional variants of the sequences disclosed in SEQ ID NO:4, SEQ ID NO:6, or SEQ ID NO:8 which differ from these sequences due to conservative amino acid substitutions. Functional variants will retain their ability to target to oil bodies in vivo.

In a further preferred embodiment, Oleo-H23P comprises the following domains; N-terminal domain is SEQ ID NO:18, hydrophobic chain HN domain 2a is SEQ ID NO:21, hydrophobic chain HN domain 3a is SEQ ID NO:22, proline knot motif is SEQ ID NO:24, hydrophobic chain HC domain 2b is SEQ ID NO:27, hydrophobic chain HC domain 3b is SEQ ID NO:28, and the C-terminal domain is SEQ ID NO:30.

In a further preferred embodiment, Oleo-H23P comprises the following domains; N-terminal domain is SEQ ID NO:33, hydrophobic chain HN domain 2a is SEQ ID NO:35, hydrophobic chain HN domain 3a is SEQ ID NO:36, proline knot motif is SEQ ID NO:38, hydrophobic chain HC domain 2b is SEQ ID NO:41, hydrophobic chain HC domain 3b is SEQ ID NO:42, and the C-terminal domain is SEQ ID NO:44.

In a further preferred embodiment, Oleo-H3P comprises the following domains; N-terminal domain is SEQ ID NO:18, hydrophobic chain HN domain 3a is SEQ ID NO:22, proline knot motif is SEQ ID NO:24, hydrophobic chain HC domain 3b is SEQ ID NO:28, and the C-terminal domain is SEQ ID NO:30.

In a further preferred embodiment, Oleo-H3P comprises the following domains; N-terminal domain is SEQ ID NO:32, hydrophobic chain HN domain 3a is SEQ ID NO:36, proline knot motif is SEQ ID NO:38, hydrophobic chain HC domain 3b is SEQ ID NO:42, and the C-terminal domain is SEQ ID NO:44.

In a further preferred embodiment, Oleo-HP comprises the following domains; N-terminal domain is SEQ ID NO:18, proline knot motif is SEQ ID NO:23, and the C-terminal domain is SEQ ID NO:30.

In a further preferred embodiment, Oleo-HP comprises the following domains; N-terminal domain is SEQ ID NO:32, proline knot motif is SEQ ID NO:37, and the C-terminal domain is SEQ ID NO:44.

Deletions using “Direction2” mode can be performed by obtaining reduced versions of the hydrophobic chains linked to the N- and C-terminal coding regions and subsequent fusion using the proline knot motif as a bridge. Specific primers annealing inside regions encoding for the hydrophobic chains can be used. All primers used should contain restriction enzymes sites in the 5′ end to allow specific fusion. In order to perform such modifications it is necessary to obtain three distinct fragments of oleosin DNA coding sequence in: (a) N-terminal plus reduced versions of hydrophobic chain HN coding sequences, (b) sequence encoding for the proline knot motif, (c) C-terminal plus reduced versions of hydrophobic chain HC coding sequences. In yet another preferred embodiment, the hydrophobic domains are reduced and the resulting modified oleosin sequences have the nucleotide sequences represented by Oleo-H12P and Oleo-H1P which are SEQ ID NO:9 or SEQ ID NO:11 respectively and the amino acid sequences represented by Oleo-H12P and Oleo-H1P which are SEQ ID NO:10 or SEQ ID NO:12 respectively. The invention further includes functional variants of the sequences disclosed in SEQ ID NO:10 or SEQ ID NO:12 which differ from these sequences due to conservative amino acid substitutions. Functional variants will retain their ability to target to oil bodies in vivo.

Ina further preferred embodiment, Oleo-H12P comprises the following domains; N-terminal domain is SEQ ID NO:17, hydrophobic chain HN domain 1a is SEQ ID NO:19, hydrophobic chain HN domain 2a is SEQ ID NO:21, proline knot motif is SEQ ID NO:24, hydrophobic chain HC domain 1b is SEQ ID NO:25, hydrophobic chain HC domain 2b is SEQ ID NO:27, and the C-terminal domain is SEQ ID NO:29.

In a further preferred embodiment, Oleo-H12P comprises the following domains; N-terminal domain is SEQ ID NO:31, hydrophobic chain HN domain 1a is SEQ ID NO:33, hydrophobic chain HN domain 2a is SEQ ID NO:35, proline knot motif is SEQ ID NO:38, hydrophobic chain HC domain 1b is SEQ ID NO:39, hydrophobic chain HC domain 2b is SEQ ID NO:41, and the C-terminal domain is SEQ ID NO:43.

In a further preferred embodiment, Oleo-H1P comprises the following domains; N-terminal domain is SEQ ID NO:17, hydrophobic chain HN domain 1a is SEQ ID NO:19, proline knot motif is SEQ ID NO:24, hydrophobic chain HC domain 1b is SEQ ID NO:25, and the C-terminal domain is SEQ ID NO:29.

In a further preferred embodiment, Oleo-H1P comprises the following domains; N-terminal domain is SEQ ID NO:31, hydrophobic chain HN domain 1a is SEQ ID NO:33, proline knot motif is SEQ ID NO:38, hydrophobic chain HC domain 1b is SEQ ID NO:39, and the C-terminal domain is SEQ ID NO:43.

(ii) Increase of Hydrophobic Domain Length

In a further preferred embodiment, the modified oleosin proteins are extended relative to their native sequence. These are expected to have an elevated affinity for an oil body if properly targeted. Increasing hydrophobic domain length can be performed by inserting DNA sequences in the regions encoding for hydrophobic chains HN and HC. It is preferred that the sequence inserted encodes for a hydrophobic peptide in order to be accommodated into the oil body triacylglycerol matrix. To maintain the structure of the modified oleosin it is also preferred to insert sequences with similar length in both hydrophobic chains. In order to perform such modifications it is necessary to obtain five different fragments of the oleosin DNA coding sequence: (a) N-terminal sequence, (b) hydrophobic extension on the HN chain, (c) hydrophobic domain (including the proline knot motif), (d) hydrophobic extension on the HC chain and (e) C-terminal sequence. The hydrophobic extension sequence can be any sequence that encodes for a peptide sufficiently hydrophobic to be inserted on the oil body. This sequence could be originated from other oleosins or transmembrane regions. In yet another preferred embodiment, the hydrophobic domains are extended and the resulting modified oleosin sequence have the nucleotide sequences represented by Oleo-double which is SEQ ID NO:13 and the amino acid sequences represented by Oleo-double which is SEQ ID NO:14. The invention further includes functional variants of the sequences disclosed in SEQ ID NO:14 which differ from these sequences due to conservative amino acid substitutions. Functional variants will retain their ability to target to oil bodies in vivo.

In a preferred embodiment, the modified oleosins with an increased hydrophobic domain are increased in size by the addition of a second copy of sub-domains 1a and 1b or 2a and 2b; or 3a and 3b; or by the addition of a second copy of sub-domains 1a 1b 2a and 2b; or 1a, 1b, 3a and 3b; or 2a, 2b, 3a and 3b; or by the addition of a second copy of sub-domains 1a, 1b, 2a, 2b, 3a and 3b; or by the addition of any of the preceding sub-domains or combinations thereof wherein such sub-domains have been extended by 1 or 2 or 3 or more amino acid residues.

In a preferred embodiment, the modified oleosins with an increased hydrophobic domain have a duplication of the hydrophobic HN and hydrophobic HC domains.

In a further preferred embodiment, Oleo-double comprises the following domains; N-terminal domain is SEQ ID NO:18, first hydrophobic chain HN domain 1a is SEQ ID NO:19; first hydrophobic chain HN domain 2a is SEQ ID NO:21; first hydrophobic chain HN domain 3a is SEQ ID NO:22; first hydrophobic chain HC domain 1b is SEQ ID NO:26, first hydrophobic chain HC domain 2b is SEQ ID NO:27; first hydrophobic chain HC domain 3b is SEQ ID NO:28; proline knot motif is SEQ ID NO:24; second hydrophobic chain HN domain 1a is SEQ ID NO:20, second hydrophobic chain HN domain 2a is SEQ ID NO:21; second hydrophobic chain HN domain 3a is SEQ ID NO:22; second hydrophobic chain HC domain 1b is SEQ ID NO:26, second hydrophobic chain HC domain 2b is SEQ ID NO:27; second hydrophobic chain HC domain 3b is SEQ ID NO:28; and the C-terminal domain is SEQ ID NO:30.

In a further preferred embodiment, Oleo-double comprises the following domains; N-terminal domain is SEQ ID NO:32, first hydrophobic chain HN domain 1a is SEQ ID NO:33; first hydrophobic chain HN domain 2a is SEQ ID NO:35; first hydrophobic chain HN domain 3a is SEQ ID NO:36; first hydrophobic chain HC domain 1b is SEQ ID NO:40, first hydrophobic chain HC domain 2b is SEQ ID NO:41; first hydrophobic chain HC domain 3b is SEQ ID NO:42; proline knot motif is SEQ ID NO:38; second hydrophobic chain HN domain 1a is SEQ ID NO:34, second hydrophobic chain HN domain 2a is SEQ ID NO:35; second hydrophobic chain HN domain 3a is SEQ ID NO:36; second hydrophobic chain HC domain 1b is SEQ ID NO:40, second hydrophobic chain HC domain 2b is SEQ ID NO:41; second hydrophobic chain HC domain 3b is SEQ ID NO:42; and the C-terminal domain is SEQ ID NO:44.

III. Uses of the Modified Oleosins

The present invention includes any and all uses of the modified oleosins described herein as well as any and all compositions of matter comprising the modified oleosins.

(i) Targeting of Heterologous Proteins

The present invention includes the use of the modified oleosin polypeptides to target a heterologous protein to an oil body as described in U.S. Pat. No. 5,650,554 which is incorporated herein by references in its entirety.

The subject method includes the steps of (a) preparing an expression cassette comprising: (1) a first nucleic acid sequence capable of regulating the transcription of (2) a second nucleic acid sequence encoding a sufficient portion of a mutant oleosin polypeptide to provide targeting to an oil body fused to (3) a third nucleic acid sequence encoding the heterologous polypeptide of interest; (b) delivering of the expression cassette into a host cell; (c) producing a transformed organism or cell population in which the chimeric gene product is expressed and (d) recovering the chimeric gene protein product through specific association with an oil body. The heterologous peptide is generally a foreign polypeptide normally not expressed in the host cell or found in association with the oil body.

The host cell may be selected from a wide range of host cells including plants, bacteria, yeasts, insects and mammals.

In one embodiment the host cell is a bacterial cell. Bacterial host cells suitable for carrying out the present invention include E. coli, B. subtilis, Salmonella typhimurium and Staphylococcus, as well as many other bacterial species well known to one of ordinary skill in the art. Representative examples of bacterial host cells include JM109 ATCC No. 53323 and DH5 (Stratagene, LaJolla, Calif.). Suitable bacterial expression vectors preferably comprise a promoter which functions in the host cell, one or more selectable phenotypic markers, and a bacterial origin of replication. Representative promoters include the LacZ, the b-lactamase (penicillinase) and lactose promoter system (see Chang et al., Nature, 1978 275:615), the trp promoter (Nichols and Yanofsky, 1983 Meth in Enzymology 101:155) and the tac promoter (Russell et al., 1982 Gene 20:231).

In another embodiment, the host cell is a yeast cell. Yeast and fungi host cells suitable for carrying out the present invention include, among others Saccharomyces cerevisae, the genera Pichia or Kluyveromyces and various species of the genus Aspergillus. Suitable expression vectors for yeast and fungi include, among others, YCₚ50 (ATCC No. 37419) for yeast, and the amdS cloning vector pV3 (Turnbull, Bio/Technology 1989 7:169). Protocols for the transformation of yeast are also well known to those of ordinary skill in the art. For example, transformation may be readily accomplished either by preparation of spheroplasts of yeast with DNA (see Hinnen et al., 1978 PNAS USA 75:1929) or by treatment with alkaline salts such as LiCl (see Itoh et al., J. Bacteriology, 1983 153:163). Transformation of fungi may also be carried out using polyethylene glycol as described by Cullen et al. Bio/Technology (1087) 5:369, 1987.

The host cell may also be a mammalian cell. Mammalian cells suitable for carrying out the present invention include, among others: COS (e.g., ATCC No. CRL 1650 or 1651), BHK (e.g., ATCC No. CRL 6281), CHO (ATCC No. CCL 61), HeLa (e.g., ATCC No. CCL 2), 293 (ATCC No. 1573) and NS-1 cells. b Suitable expression vectors for directing expression in mammalian cells generally include a promoter, as well as other transcriptional and translational control sequences. Suitable promoters include PMSG, pSVL, SV40, pCH 110, MMTV, metallothionein-1, adenovirus Ela, CMV, immediate early, immunoglobulin heavy chain promoter and enhancer, and RSV-LTR. Protocols for the transfection of mammalian cells are well known to those of ordinary skill in the art. Representative methods include calcium phosphate mediated electroporation, retroviral, and protoplast fusion-mediated transfection (see Sambrook et al., 1989 Molecular Cloning a Laboratory Manual, 2nd Edition, Cold Spring Harbour Laboratory Press).

The host cell may also be an insect cell. Insect cells suitable for carrying out the present invention include cells and cell lines from Bombyx or Spodotera species. Suitable expression vectors for directing expression in insect cells include Baculoviruses such as the Autographa california nuclear polyhedrosis, virus (Miller et al. 1987, in Genetic Engineering, Vol. 8 ed. Setler, J. K. et al., Plenum Press, New York) and the Bombyx mori nuclear polyhedrosis virus (Maeda et al., 1985 Nature 315:592).

In a preferred embodiment, the host cell is a plant and the chimeric product is expressed and translocated to the oil bodies of the seed. The use of plants to produce proteins of interest allows exploitation of the ability of plants to capture energy and limited nutrient input to make proteins. The scale and yield of material afforded by production in plants allows adaptation of the technology for use in the production of a variety of polypeptides of commercial interest. The plant may be selected from various plant families including Brassicaceae, Compositae, Euphorbiaceae, Leguminosae, Linaceae, Malvaceae, Umbilliferae and Graininae. The plant may be selected from various plant species including rapeseed (Brassica spp.), linseed/flax (Linum usitatissimum), safflower (Carthamus tinctorius), sunflower (Helianthus annuus), maize (Zea mays), soybean (Glycine max), mustard (Brassica spp. and Sinapis alba), crambe, (Crambe abyssinica), eruca (Eruca sativa), oil palm (Elaeis guineeis), cottonseed (Gossypium spp.), groundnut (Arachis hypogaea), coconut (Cocus nucifera), castor bean (Ricinus communis), coriander (Coriandrum sativum), squash, (Cucurbita maxima), Brazil nut (Bertholletia excelsa) and jojoba (Simmondsia chinensis).

The use of a modified oleosin protein as a carrier or targeting means provides a simple mechanism to recover proteins. The chimeric protein associated with the oil body may be separated away from the bulk of cellular components in a single step by isolation of the oil body fraction using for example centrifugation size exclusion or floatation. The invention contemplates the use of heterologous proteins, including enzymes, therapeutic proteins, diagnostic proteins and the like fused to modified oleosins and associated with oil bodies. Association of the protein with the oil body allows subsequent recovery of the protein by simple means (centrifugation and floatation).

In accordance with further embodiments of the invention the heterologous proteins fused to modified oleosin proteins associated with isolated oil bodies may be released and separated from the oleosin. Accordingly an expression cassette may be prepared which comprises a first nucleic acid sequence capable of regulating the transcription of a second nucleic acid sequence encoding a sufficient portion of a modified oleosin to provide targeting to an oil body and fused to this second nucleic acid sequence a nucleic acid sequence encoding a amino acid sequence cleavable by a specific protease or by chemical treatment, and a third nucleic acid sequence encoding the heterologous polypeptide; such that the protein of interest can be cleaved from the isolated oil body fraction by the specific chemical treatment or protease.

According to one embodiment of the invention the expression cassette is introduced into a host cell in a form where the expression cassette is stably incorporated into the genome of the host cell. Accordingly it is apparent that one may also introduce the expression cassette as part of a recombinant nucleic acid sequence capable of replication and or expression in the host cell without the need to become integrated into the host chromosome.

In an alternative embodiment of the invention nucleic acid is stably incorporated into the genome of the host cell by homologous recombination. Examples of gene targeting by homologous recombination have been described for various cell types including mammalian cells (Mansour et al., 1988 Nature, 336, 348-352) and plant cells (Miao and Lam, 1995 Plant Journal, 7: 359-365). Introduction into the host cell genome of the protein of interest may be accomplished by homologous recombination of the protein of interest in such a fashion that upon recombination an expression cassette is generated which will generally include, in the 5′-3′ direction of transcription, a first nucleic acid sequence comprising a transcriptional and translational regulatory region capable of expression in the host cell, a second nucleic acid sequence encoding a fusion protein comprising a sufficient portion of a modified oleosin protein to provide targeting to an oil body and a heterologous protein, and a transcriptional and translational termination region functional in plants.

For production of recombinant protein modified oleosin fusions in heterologous systems such as animal, insect or microbial species, promoters would be chosen for maximal expression in said cells, tissues or organs to be used for recombinant protein production. The invention is contemplated for use in a variety of organisms which can be genetically altered to express foreign proteins including animals, especially those producing milk such as cattle and goats, invertebrates such as insects, specifically insects that can be reared on a large scale, more specifically those insects which can be infected by recombinant baculoviruses that have been engineered to express oleosin fusion proteins, fungal cells such as yeasts and bacterial cells. Promoter regions highly active in viruses, microorganisms, fungi, insects and animals are well described in the literature and may be commercially available or can be obtained by standard methods known to a person skilled in the art. It is preferred that all of the transcriptional and translational functional elements of the initiation control region are derived from or obtained from the same gene.

For those applications where expression of the recombinant protein is derived from extrachromosomal elements, one may chose a replicon capable of maintaining a high copy number to maximize expression. Alternatively or in addition to high copy number replicons, one may further modify the recombinant nucleic acid sequence to contain specific transcriptional or translation enhancement sequences to assure maximal expression of the foreign protein in host cells.

The level of transcription should be sufficient to provide an amount of RNA capable of resulting in a modified seed, cell, tissue, organ or organism. The term “modified” as used in the preceding sentence is meant a detectably different phenotype of a seed, cell, tissue, organ or organism in comparison to the equivalent non-transformed material, for example one not having the expression cassette in question in its genome. It is noted that the RNA may also be an “antisense RNA” capable of altering a phenotype by inhibition of the expression of a particular gene.

The recombinant expression vectors and chimeric nucleic acid sequences of the present invention may be prepared in accordance with methodologies well known to those skilled in the art of molecular biology. Such preparation will typically involve the bacterial species Escherichia coli as an intermediary cloning host. The preparation of the E. coli vectors as well as the plant transformation vectors may be accomplished using commonly known techniques such as restriction digestion, ligation, gel ectrophoresis, DNA sequencing, the Polymerase Chain Reaction (PCR) and other methodologies. A wide variety of cloning vectors is available to perform the necessary steps required to prepare a recombinant expression vector. Among the vectors with a replication system functional in E. coli, are vectors such as pBR322, the pUC series of vectors, the M13mp series of vectors, pBluescript etc. Typically, these cloning vectors contain a marker allowing selection of transformed cells. Nucleic acid sequences may be introduced in these vectors, and the vectors may be introduced in E. coli grown in an appropriate medium. Recombinant expression vectors may readily be recovered from cells upon harvesting and lysing of the cells. Further, general guidance with respect to the preparation of recombinant vectors may be found in, for example: Sambrook et al., Molecular Cloning, a Laboratory Manual, Cold Spring Harbor Laboratory Press, 1989, Vol. 3.

The mode by which the modified oleosin protein and the protein to be expressed are fused can be either a N-terminal, C-terminal or internal fusion. The choice is dependent upon the application. For example, C-terminal fusions can be made as follows: A genomic clone of an oleosin protein gene preferably containing at least 100 bp 5′ to the translational start is cloned into a plasmid vehicle capable of replication in a suitable bacterial host (e.g., pUC or pBR322 in E. coli). A restriction site is located in the region encoding the hydrophilic C-terminal portion of gene. In a plant oleosin protein of approximately 18 KDa, such as the Arabidopsis oleosin, this region stretches typically from codons 125 to the end of the clone. The ideal restriction site is unique, but this is not absolutely essential. If no convenient restriction site is located in this region, one may be introduced by site-directed mutagenesis. The only major restriction on the introduction of this site is that it must be placed 5′ to the translational stop signal of the oleosin protein clone.

With this altered clone in place, a synthetic oligonucleotide adapter may be produced which contains coding sequence for a protease recognition site or a multimer thereof. This, may be for example the recognition site for the protease collagenase. The adaptor would be synthesized in such a way as to provide a 4-base overhang at the 5′ end compatible with the restriction site at the 3′ end of the modified oleosin protein clone, a 4-base overhang at the 3′ end of the adaptor to facilitate ligation to the foreign peptide coding sequence and additional bases, if needed, to ensure no frame shifts in the transition between the modified oleosin protein coding sequence, the protease recognition site and the foreign peptide coding sequence. The final ligation product will contain an almost complete modified oleosin protein gene, coding sequence for collagenase recognition motif and the desired polypeptide coding region all in a single reading frame.

A similar approach is used for N-terminal fusions. The hydrophilic N-terminal end of modified oil body proteins permits the fusion of peptides to the N-terminal while still assuring that the foreign peptide would be retained on the outer surface of the oil body. This configuration can be constructed from similar starting materials as used for C-terminal fusions, but requires the identification of a convenient restriction site close to the translational start of the modified oleosin protein gene. A convenient site may be created in many plant oleosin protein genes without any alteration in coding sequence by the introduction of a single base change just 5′ to the start codon (ATG). In plant oleosin proteins thus far studied, the second amino acid is alanine whose codon begins with a “G”. A-C transition at that particular “G” yields a Nco I site.

The coding sequence for the foreign peptide may require preparation which will allow its ligation directly into the introduced restriction site. For example, introduction of a coding sequence into the Nco I site introduced into the oleosin protein coding sequences described above may require the generation of compatible ends. This may typically require a single or two-base modification by site-directed mutagenesis to generate an Nco I site around the translational start of the foreign peptide. This peptide is then excised from its cloning vehicle using Nco I and a second enzyme which cuts close to the translational stop of the target. Again, using the methods described above, a second convenient site can be introduced by site-directed mutagenesis. It has been suggested by Qu and Huang (1990) J Biol Chem. 265(4):2238-43. that the N-terminal methionine might be removed during processing of the plant oleosin proteins protein in vivo and that the alanine immediately downstream of this might be acylated. To account for this possibility, it may be necessary to retain the Met-Ala sequence at the N-terminal end of the protein. This is easily accomplished using a variety of strategies which introduce a convenient restriction site into the coding sequence in or after the Ala codon.

The resultant constructs from these N-terminal fusions may contain an oleosin protein promoter sequence, an in-frame fusion in the first few codons of the oleosin protein gene of a high value peptide coding sequence with its own ATG as start signal if necessary and the remainder of the oleosin protein gene and terminator.

In principle any desired protein or peptide may be produced using this technology and oil bodies comprising these recombinant proteins may be incorporated in the emulsions of the present invention. The invention is not limited by the source or the use of the heterologous polypeptide.

The nucleic acid sequence encoding the heterologous polypeptide of interest may be synthetic, naturally derived, or a combination thereof. Dependent upon the nature or source of the nucleic acid encoding the polypeptide of interest, it may be desirable to synthesize the nucleic acid sequence with codons that represent the preference of the organism in which expression takes place. For expression in plant species, one may employ plant preferred codons. The plant preferred codons may be determined from the codons of highest frequency in the proteins expressed in the largest amount in the particular plant species of interest as a host plant.

In order to produce cells, plants and seeds comprising the recombinant fusion protein, the chimeric nucleic acid sequence is incorporated in a recombinant expression vector. Accordingly, provided herein are recombinant expression vectors comprising the nucleic acids provided herein suitable for expression of the modified oleosin polypeptides, suitable for the selected cell. The term “suitable for expression in the selected cell” means that the recombinant expression vector contains all nucleic acid sequences required to ensure expression in the selected cell.

Accordingly, the recombinant expression vectors further contain regulatory nucleic acid sequences selected on the basis of the cell which is used for expression and ensuring initiation and termination of transcription operatively linked to the nucleic acid sequence encoding the modified oleosin. Regulatory nucleic acid sequences include promoters, enhancers, silencing elements, ribosome binding sites, Shine-Dalgarno sequences, introns and other expression elements. “Operatively linked” is intended to mean that the nucleic acid sequences comprising the regulatory regions linked to the nucleic acid sequences encoding the modified oleosin expression in the cell. A typical nucleic acid construct comprises in the 5′ to 3′ direction a promoter region capable of directing expression, a coding region comprising the modified oleosin polypeptide and a termination region functional in the selected cell.

The selection of regulatory sequences will depend on the organism and the cell type in which the modified oleosin is expressed, and may influence the expression levels of the polypeptide. Regulatory sequences are art-recognized and selected to direct expression of the modified oleosin in the cell.

Promoters that may be used in bacterial cells include the lac promoter (Blackman et al., 1978 Cell 13: 65-71), the trp promoter (Masuda et al., 1996 Protein Eng 9: 101-106) and the T7 promoters (Studier et al., 1986 J. Mol. Biol. 189: 113-130). Promoters functional in plant cells that may be used herein include constitutive promoters such as the 35S CaMV promoter (Rothstein et al., 1987 Gene 53: 153-161) the actin promoter (McElroy et al., 1990 Plant Cell 2: 163-171) and the ubiquitin promoter (European Patent Application 0 342 926). Other promoters are specific to certain tissues or organs (for example, roots, leaves, flowers or seeds) or cell types (for example, leaf epidermal cells, mesophyll cells or root cortex cells) and or to certain stages of plant development. Timing of expression may be controlled by selecting an inducible promoter, for example the PR-a promoter described in U.S. Pat. No. 5,614,395. Selection of the promoter therefore depends on the desired location and timing of the accumulation of the desired polypeptide. In a particular embodiment, the modified oleosin expressed in a seed cell and seed specific promoters are utilized. Seed specific promoters that may be used herein include for example the phaseolin promoter (Sengupta-Gopalan et al., 1985 Proc. Natl. Acad. Sci. USA: 82 3320-3324), and the Arabidopsis 18 kDa oleosin promoter (van Rooijen et al., 1992 Plant. Mol. Biol. 18: 1177-1179). New promoters useful in various plant cell types are constantly discovered. Numerous examples of plant promoters may be found in Ohamuro et al. (Biochem of Pl., 1989 15: 1-82).

Genetic elements capable of enhancing expression of the polypeptide may be included in the expression vectors. In plant cells these include for example, the untranslated leader sequences from viruses such as the AMV leader sequence (Jobling and Gehrke, 1987 Nature 325: 622-625) and the intron associated with the maize ubiquitin promoter (See: U.S. Pat. No. 5,504,200).

Transcriptional terminators are generally art recognized and besides serving as a signal for transcription termination serve as a protective element serving to extend the mRNA half-life (Guarneros et al., 1982 Proc. Natl. Acad. Sci. USA 79: 238-242). In nucleic acid sequences for the expression in plant cells, the transcriptional terminator typically is from about 200 nucleotide to about 1000 nucleotides in length. Terminator sequences that may be used herein include for example, the nopaline synthase termination region (Bevan et al., 1983 Nucl. Acid. Res. 11: 369-385), the phaseolin terminator (van der Geest et al., 1994 Plant J. 6: 413-423), the terminator for the octopine synthase gene of Agrobacterium tumefaciens or other similarly functioning elements. Transcriptional terminators can be obtained as described by An (1987) Methods in Enzym. 153: 292. The selection of the transcriptional terminator may have an effect on the rate of transcription.

Accordingly, provided herein are chimeric nucleic acid sequences encoding a recombinant fusion polypeptide. In one embodiment, said nucleic acid

1) a first nucleic acid sequence capable of regulating the transcription in said cell;

2) a second nucleic acid sequence, wherein said second sequence encodes a fusion polypeptide and comprises (i) a nucleic acid sequence encoding a modified oleosin peptide of the invention to provide targeting of the fusion polypeptide to a lipid phase linked in reading frame to (ii) a nucleic acid sequence encoding the heterologous polypeptide; and

3) a third nucleic acid sequence encoding a termination region functional in the host cell.

The recombinant expression vector further may contain a marker gene. Marker genes that may be used in accordance with the present invention include all genes that allow the distinction of transformed cells from non-transformed cells including all selectable and screenable marker genes. A marker may be a resistance marker such as an antibiotic resistance marker against for example kanamycin, ampicillin, G418, bleomycin hygromycin, chloramphenicol which allows selection of a trait by chemical means or a tolerance marker against for example a chemical agent such as the normally phytotoxic sugar mannose (Negrotto et al., 2000 Plant Cell Rep. 19: 798-803). In plant recombinant expression vectors herbicide resistance markers may conveniently be used for example markers conferring resistance against glyphosate (U.S. Pat. Nos. 4,940,935 and 5,188,642) or phosphinothricin (White et al., 1990 Nucl. Acids Res. 18: 1062; Spencer et al., 1990 Theor. Appl. Genet. 79: 625-631). Resistance markers to a herbicide when linked in close proximity to the redox protein or oil-body-targeting-protein may be used to maintain selection pressure on a population of plant cells or plants for those plants that have not lost the protein of interest. Screenable markers that may be employed to identify transformants through visual observation include beta-glucuronidase (GUS) (see US Patents U.S. Pat. No. 5,268,463 and U.S. Pat. No. 5,599,670) and green fluorescent protein (GFP) (Niedz et al., 1995 Plant Cell Rep. 14: 403).

Recombinant expression vectors suitable for the introduction of nucleic acid sequences in plant cells include Agrobacterium and Rhizobium based vectors such as the Ti and Ri plasmids. Agrobacterium based vectors typically carry at least one T-DNA border sequence and include vectors such pBIN 19 (Bevan, 1984 Nucl Acids Res. Vol. 12, 22:8711-8721) and other binary vector systems (for example: U.S. Pat. No. 4,940,838).

In accordance with the present invention, the recombinant expression vectors are introduced into the cell that is selected and the selected cells are grown to produce the modified oleosin protein in a progeny cell.

Methodologies to introduce recombinant expression vectors into a cell also referred to herein as “transformation” are well known to the art and vary depending on the cell type that is selected. General techniques to transfer the recombinant expression vectors into the cell include electroporation; chemically mediated techniques, for example CaCl2 mediated nucleic acid uptake; particle bombardment (biolistics); the use of naturally infective nucleic acid sequences for example virally derived nucleic acid sequences or when plant cells are used Agrobacterium or Rhizobium derived nucleic acid sequences; PEG mediated nucleic acid uptake, microinjection, and the use of silicone carbide whiskers (Kaeppler et al., 1990 Plant Cell Rep. 9:415-418) all of which may be used herein.

Introduction of the recombinant expression vector into the cell may result in integration of its whole or partial uptake into host cell genome including the chromosomal DNA or the plastid genome. Alternatively the recombinant expression vector may not be integrated into the genome and replicate independently of the host cell's genomic DNA. Genomic integration of the nucleic acid sequence is typically used as it will allow for stable inheritance of the introduced nucleic acid sequences by subsequent generations of cells and the creation of cell, plant or animal lines.

Particular embodiments involve the use of plant cells. Particular plant cells used herein include cells obtainable from Brazil nut (Betholletia excelsa); castor (Riccinus communis); coconut (Cocus nucifera); coriander (Coriandrum sativum); cotton (Gossypium spp.); groundnut (Arachis hypogaea); jojoba (Simmondsia chinensis); linseed/flax (Linum usitatissimum); maize (Zea mays); mustard (Brassica spp. and Sinapis alba); oil palm (Elaeis guineeis); olive (Olea europaea); rapeseed (Brassica spp.); safflower (Carthamus tinctorius); soybean (Glycine max); squash (Cucurbita maxima); barley (Hordeum vulgare); wheat (Traeticum aestivum) and sunflower (Helianthus annuus).

Transformation methodologies for dicotelydenous plant species are well known. Generally Agrobacterium mediated transformation is utilized because of its high efficiency as well as the general susceptibility by many, if not all dicotelydenous plant species. Agrobacterium transformation generally involves the transfer of a binary vector (e.g. pBIN19) comprising the DNA of interest to an appropriate Agrobacterium strain (e.g. CIB542) by for example tri-parental mating with an E. coli strain carrying the recombinant binary vector and an E. coli strain carrying a helper plasmid capable of mobilization of the binary vector to the target Agrobacterium strain, or by DNA transformation of the Agrobacterium strain (Hofgen et al. Nucl. Acids. Res., 1988 16: 9877. Other transformation methodologies that may be used to transform dicotelydenous plant species include biolistics (Sanford, 1988 Trends in Biotechn. 6: 299-302); electroporation (Fromm et al., 1985 Proc. Natl. Acad. Sci. USA 82: 5824-5828); PEG mediated DNA uptake (Potrykus et al., 1985 Mol. Gen. Genetics 199: 169-177); microinjection (Reich et al., 1986 Bio/Techn. 4: 1001-1004) and silicone carbide whiskers (Kaeppler et al., 1990 Plant Cell Rep. 9: 415-418). The exact transformation methodologies typically vary somewhat depending on the plant species that is used.

In a particular embodiment the oil bodies are obtained from safflower and the recombinant proteins are expressed in safflower. Safflower transformation has been described by Baker and Dyer (1996 Plant Cell Rep. 16: 106-110.)

Monocotelydenous plant species may now also be transformed using a variety of methodologies including particle bombardment (Christou et al., 1991 Biotechn. 9: 957-962; Weeks et al., 1993 Plant Physiol. 102: 1077-1084; Gordon-Kamm et al., 1990 Plant Cell 2: 603-618) PEG mediated DNA uptake (EP 0 292 435; 0 392 225) or Agrobacterium-mediated transformation (Goto-Fumiyuki et al., 1999 Nature-Biotech. 17 (3):282-286).

Plastid transformation is described in U.S. Pat. Nos. 5,451,513; 5,545,817 and 5,545,818; and PCT Patent Applications 95/16783; 98/11235 and 00/39313) Basic chloroplast transformation involves the introduction of cloned plastid DNA flanking a selectable marker together with the nucleic acid sequence of interest into a suitable target tissue using for example biolistics or protoplast transformation. Selectable markers that may be used include for example the bacterial aadA gene (Svab et al., 1993 Proc. Natl. Acad. Sci. USA 90: 913-917). Plastid promoters that may be used include for example the tobacco clpP gene promoter (PCT Patent Application 97/06250).

In another embodiment, the invention chimeric nucleic acid contructs provided herein are directly transformed into the plastid genome. Plastid transformation technology is described extensively in U.S. Pat. Nos. 5,451,513, 5,545,817, 5,545,818 and 5,576,198; in PCT application nos. WO 95/16783 and WO 97/32977; and in McBride et. al., 1994 Proc Natl Acad Sci USA 91: 7301-7305, the entire disclosures of all of which are hereby incorporated by reference. In one embodiment, plastid transformation is achieved via biolistics, first carried out in the unicellular green alga Chlamydomonas reinhardtii (Boynton et al., 1988 Science 240:1534-1537)) and then extended to Nicotiana tabacum (Svab et al., 1990 Proc Natl Acad Sci USA 87:8526-8530), combined with selection for cis-acting antibiotic resistance loci (spectinomycin or streptomycin resistance) or complementation of non-photosynthetic mutant phenotypes.

In another embodiment, tobacco plastid transformation is carried out by particle bombardment of leaf or callus tissue, or polyethylene glycol (PEG)-mediated uptake of plasmid DNA by protoplasts, using cloned plastid DNA flanking a selectable antibiotic resistance marker. For example, 1 to 1.5 kb flanking regions, termed targeting sequences, facilitate homologous recombination with the plastid genome and allow the replacement or modification of specific regions of the 156 kb tobacco plastid genome. In one embodiment, point mutations in the plastid 16S rDNA and rps12 genes conferring resistance to spectinomycin and/or streptomycin can be utilized as selectable markers for transformation (Svab et al., 1990 Proc Natl Acad Sci USA 87:8526-8530; Staub et al., 1992 Plant Cell 4:39-45 the entire disclosures of which are hereby incorporated by reference), resulting in stable homoplasmic transformants at a frequency of approximately one per 100 bombardments of target leaves. The presence of cloning sites between these markers allows creation of a plastid targeting vector for introduction of foreign genes (Staub et al., 1993 EMBO J 12:601-606, the entire disclosure of which is hereby incorporated by reference). In another embodiment, substantial increases in transformation frequency can be obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab et al., 1993 Proc Natl Acad Sci USA 90: 913-917, the entire disclosure of which is hereby incorporated by reference). This marker has also been used successfully for high-frequency transformation of the plastid genome of the green alga Chlamydomonas reinhardtii (Goldschmidt-Clermont, M., 1991 Nucl Acids Res 19, 4083-4089, the entire disclosure of which is hereby incorporated by reference). In other embodiments, plastid transformation of protoplasts from tobacco and the moss Physcomitrella can be attained using PEG-mediated DNA uptake (O'Neill et al., 1993 Plant J 3:729-738; Koop et al., 1996 Planta 199:193-201, the entire disclosures of which are hereby incorporated by reference).

Both particle bombardment and protoplast transformation are also contemplated for use herein. Plastid transformation of oilseed plants has been successfully carried out in the genera Arabidopsis and Brassica (Sikdar et al, 1998 Plant Cell Rep 18:20-24; PCT Application WO 00/39313, the entire disclosures of which are hereby incorporated by reference).

A chimeric nucleic sequence construct is inserted into a plastid expression cassette including a promoter capable of expressing the construct in plant plastids. A particular promoter capable of expression in a plant plastid is, for example, a promoter isolated from the 5′ flanking region upstream of the coding region of a plastid gene, which may come from the same or a different species, and the native product of which is typically found in a majority of plastid types including those present in non-green tissues. Gene expression in plastids differs from nuclear gene expression and is related to gene expression in prokaryotes (Stern et al., 1997 Trends in Plant Sci 2:308-315, the entire disclosure of which is hereby incorporated by reference).

Plastid promoters generally contain the −35 and −10 elements typical of prokaryotic promoters, and some plastid promoters called PEP (plastid-encoded RNA polymerase) promoters are recognized by an E. coli-like RNA polymerase mostly encoded in the plastid genome, while other plastid promoters called NEP promoters are recognized by a nuclear-encoded RNA polymerase. Both types of plastid promoters are suitable for use herein. Examples of plastid promoters include promoters of clpP genes such as the tobacco clpP gene promoter (WO 97/06250, the entire disclosure of which is hereby incorporated by reference) and the Arabidopsis clpP gene promoter (U.S. application Ser. No. 09/038,878, the entire disclosure of which is hereby incorporated by reference). Another promoter capable of driving expression of a chimeric nucleic acid construct in plant plastids comes from the regulatory region of the plastid 16S ribosomal RNA operon (Harris et al., 1994 Microbiol Rev 58:700-754; Shinozaki et al., 1986 EMBO J 5:2043-2049, the entire disclosures of both of which are hereby incorporated by reference). Other examples of promoters capable of driving expression of a nucleic acid construct in plant plastids include a psbA promoter or am rbcL promoter. A plastid expression cassette preferably further includes a plastid gene 3′ untranslated sequence (3′ UTR) operatively linked to a chimeric nucleic acid construct of the present invention. The role of untranslated sequences is preferably to direct the 3′ processing of the transcribed RNA rather than termination of transcription. An exemplary 3′ UTR is a plastid rps16 gene 3′ untranslated sequence, or the Arabidopsis plastid psbA gene 3′ untranslated sequence. In a further embodiment, a plastid expression cassette includes a poly-G tract instead of a 3′ untranslated sequence. A plastid expression cassette also preferably further includes a 5′ untranslated sequence (5′ UTR) functional in plant plastids, operatively linked to a chimeric nucleic acid construct provided herein.

A plastid expression cassette is contained in a plastid transformation vector, which preferably further includes flanking regions for integration into the plastid genome by homologous recombination. The plastid transformation vector may optionally include at least one plastid origin of replication. The present invention also encompasses a plant plastid transformed with such a plastid transformation vector, wherein the chimeric nucleic acid construct is expressible in the plant plastid. Also encompassed herein is a plant or plant cell, including the progeny thereof, including this plant plastid. In a particular embodiment, the plant or plant cell, including the progeny thereof, is homoplasmic for transgenic plastids.

Other promoters capable of driving expression of a chimeric nucleic acid construct in plant plastids include transactivator-regulated promoters, preferably heterologous with respect to the plant or to the subcellular organelle or component of the plant cell in which expression is effected. In these cases, the DNA molecule encoding the transactivator is inserted into an appropriate nuclear expression cassette which is transformed into the plant nuclear DNA. The transactivator is targeted to plastids using a plastid transit peptide. The transactivator and the transactivator-driven DNA molecule are brought together either by crossing a selected plastid-transformed line with and a transgenic line containing a DNA molecule encoding the transactivator supplemented with a plastid-targeting sequence and operably linked to a nuclear promoter, or by directly transforming a plastid transformation vector containing the desired DNA molecule into a transgenic line containing a chimeric nucleic acid construct encoding the transactivator supplemented with a plastid-targeting sequence operably linked to a nuclear promoter. If the nuclear promoter is an inducible promoter, in particular a chemically inducible embodiment, expression of the chimeric nucleic acid construct in the plastids of plants is activated by foliar application of a chemical inducer. Such an inducible transactivator-mediated plastid expression system is preferably tightly regulatable, with no detectable expression prior to induction and exceptionally high expression and accumulation of protein following induction.

A particular transactivator is, for example, viral RNA polymerase. Particular promoters of this type are promoters recognized by a single sub-unit RNA polymerase, such as the T7 gene 10 promoter, which is recognized by the bacteriophage T7 DNA-dependent RNA polymerase. The gene encoding the T7 polymerase is preferably transformed into the nuclear genome and the T7 polymerase is targeted to the plastids using a plastid transit peptide. Promoters suitable for nuclear expression of a gene, for example a gene encoding a viral RNA polymerase such as the T7 polymerase, are described above and elsewhere in this application. Expression of chimeric nucleic acid constructs in plastids can be constitutive or can be inducible, and such plastid expression can be also organ- or tissue-specific. Examples of various expression systems are extensively described in WO 98/11235, the entire disclosure of which is hereby incorporated by reference. Thus, in one aspect, the present invention utilizes coupled expression in the nuclear genome of a chloroplast-targeted phage T7 RNA polymerase under the control of the chemically inducible PR-1a promoter, for example of the PR-1 promoter of tobacco, operably linked with a chloroplast reporter transgene regulated by T7 gene 10 promoter/terminator sequences, for example as described in as in U.S. Pat. No. 5,614,395 the entire disclosure of which is hereby incorporated by reference. In another embodiment, when plastid transformants homoplasmic for the maternally inherited TR or NTR genes are pollinated by lines expressing the T7 polymerase in the nucleus, F1 plants are obtained that carry both transgene constructs but do not express them until synthesis of large amounts of enzymatically active protein in the plastids is triggered by foliar application of the PR-1a inducer compound benzo(1,2,3)thiadiazole-7-carbothioic acid S-methyl ester (BTH).

Following transformation the cells are grown, typically in a selective medium allowing the identification of transformants. Cells may be harvested in accordance with methodologies known to the art. In order to further associate the oil bodies containing the modified oleosin-recombinant fusion protein with a second protein which has an affinity for the modified oleosin-recombinant fusion protein, the integrity of cells may be disrupted using any physical, chemical or biological methodology capable of disrupting the cells' integrity. These methodologies are generally cell-type dependent and known to the skilled artisan. Where plants are employed they may be regenerated into mature plants using plant tissue culture techniques generally known to the skilled artisan. Seeds may be harvested from mature transformed plants and used to propagate the plant line. Plants may also be crossed and in this manner, contemplated herein is the breeding of cells lines and transgenic plants that vary in genetic background.

The invention also includes oil bodies comprising the recombinant fusion proteins of the invention. In order to prepare oil bodies from plant seeds, plants are grown and allowed to set seed in accordance with common agricultural practices. Thus, the present invention also provides seeds comprising oil bodies, wherein said oil bodies comprise a recombinant fusion protein of the invention. Upon harvesting the seed and, if necessary the removal of large insoluble materials such as stones or seed hulls, by for example sieving or rinsing, any process suitable for the isolation of oil bodies from seeds may be used herein. A typical process involves grinding of the seeds followed by an aqueous extraction process.

Seed grinding may be accomplished by any comminuting process resulting in a substantial disruption of the seed cell membrane and cell walls without compromising the structural integrity of the oil bodies present in the seed cell. Suitable grinding processes in this regard include mechanical pressing and milling of the seed. Wet milling processes such as decribed for cotton (Lawhon et al., 1977 J. Am. Oil Chem. Soc. 63: 533-534) and soybean (U.S. Pat. No. 3,971,856; Carter et al., 1974 J. Am. Oil Chem. Soc. 51: 137-141) are particularly useful in this regard. Suitable milling equipment capable of industrial scale seed milling include colloid mills, disc mills, pin mills, orbital mills, IKA mills and industrial scale homogenizers. The selection of the milling equipment will depend on the seed, which is selected, as well as the throughput requirement.

Solid contaminants such as seed hulls, fibrous materials, undissolved carbohydrates, proteins and other insoluble contaminants are subsequently preferably removed from the ground seed fraction using size exclusion based methodologies such as filtering or gravitational based methods such as a centrifugation based separation process. Centrifugation may be accomplished using for example a decantation centrifuge such as a HASCO 200 2-phase decantation centrifuge or an NX310B (Alpha Laval). Operating conditions are selected such that a substantial portion of the insoluble contaminants and sediments and may be separated from the soluble fraction.

Following the removal of insolubles the oil body fraction may be separated from the aqueous fraction. Gravitational based methods as well as size exclusion based technologies may be used. Gravitational based methods that may be used include centrifugation using for example a tubular bowl centrifuge such as a Sharples AS-16 or AS-46 (Alpha Laval), a disc stack centrifuge or a hydrocyclone, or separation of the phases under natural gravitation. Size exclusion methodologies that may be used include membrane ultra filtration and crossflow microfiltration.

Separation of solids and separation of the oil body phase from the aqueous phase may also be carried out concomitantly using gravity based separation methods or size exclusion based methods.

The oil body preparations obtained at this stage in the process are generally relatively crude and depending on the application of the oil bodies, it may be desirable to remove additional contaminants. Any process capable of removing additional seed contaminants may be used in this regard. Conveniently the removal of these contaminants from the oil body preparation may be accomplished by resuspending the oil body preparation in an aqueous phase and re-centrifuging the resuspended fraction, a process referred to herein as “washing the oil bodies”. The washing conditions selected may vary depending on the desired purity of the oil body fractions. For example where oil bodies are used in pharmaceutical compositions, generally a higher degree of purity may be desirable than when the oil bodies are used in food preparations. The oil bodies may be washed one or more times depending on the desired purity and the ionic strength, pH and temperature may all be varied. Analytical techniques may be used to monitor the removal of contaminants. For example SDS gel electrophoresis may be employed to monitor the removal of seed proteins.

The entire oil body isolation process may be performed in a batch wise fashion or continuous flow. In a particular embodiment, industrial scale continuous low processes are utilized.

Through the application of these and similar techniques the skilled artisan is able to obtain oil bodies from any cell comprising oil bodies. The skilled artisan will recognize that generally the process will vary somewhat depending on the cell type that is selected. However, such variations may be made without departing from the scope and spirit of the present invention.

(ii) Compositions

The present invention includes compositions comprising modified oleosins of the invention. Such compositions include oil bodies, plant seeds and plants comprising a modified oleosin of the invention. The oil bodies, plants and plant seeds can be prepared as described in Section III(i).

### EXAMPLES

The following examples are offered by way of illustration and not by limitation.

### Example 1

Reduction of the Hydrophobic Domain in Regions Flanking N- and C-Terminal Domains.

The hydrophobic domain from Oleo-FL can be reduced in the regions flanking the N- and C-terminal domain as described in FIG. 8c (reduction in “Direction 1”).

Construction of OleoH23P

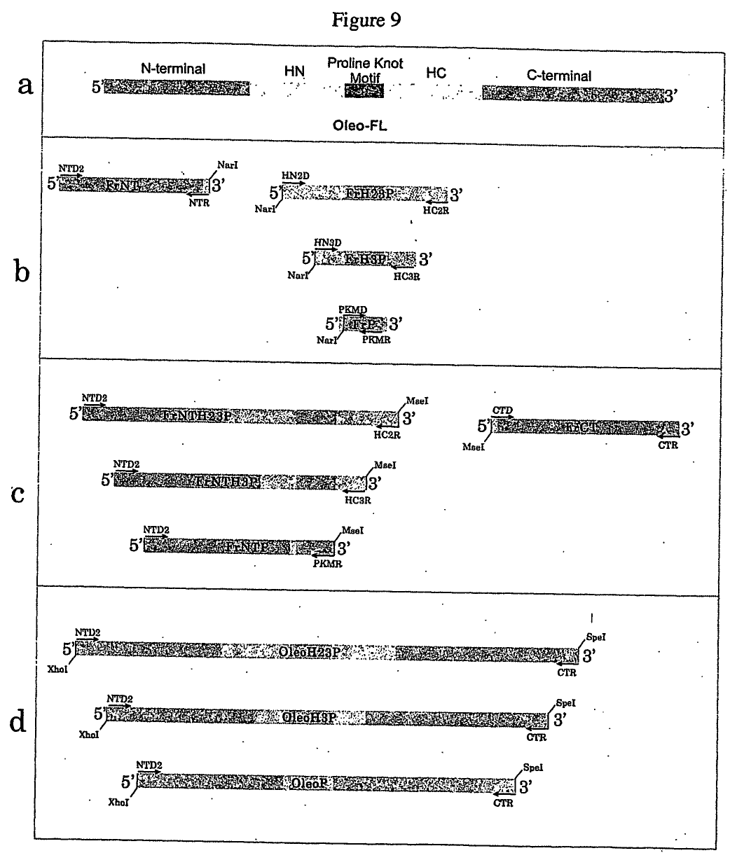

The fragment coding for the N-terminal domain (FrNT) is amplified using the forward primer NTD2 (5′-TATTCTCGAGCCATGGCGGATACTGCTAGAGG-3′)—SEQ ID NO:45 containing XhoI and NcoI restriction sites (underlined) and the reverse primer NTR (5′-CAGTGGCGCCTTTAGCAATCTGTCTAGAC-3′)—SEQ ID NO:46 containing the NarI restriction site (underlined) using Oleo-FL cDNA as template. The fragment FrH23P is amplified using the forward primer HN2D (5′-CAGCTGGTGGTGGCGCCTTGTTCTCTCC-3′)—SEQ ID NO:47 containing NarI restriction site (underlined) and the reverse primer HC2R (5′-TTATATTAAAAATGCCAAACCCTCCAG-3′)—SEQ ID NO:48 containing the MseI restriction site (underlined) using Oleo-FL cDNA as template. (FIG. 9b).

These PCR fragments are purified and digested with the enzyme NarI creating cohesive ending in the 3′ end of the fragment FrNT and in the 5′ end of the fragment FrH23P. The fragments are purified from the digestion reaction. The FrH23P fragment is then ligated to the FrNT.

The fragment FrNTH23P is amplified using the forward primer NTD2 (5′-TATTCTCGAGCCATGGCGGATACTGCTAGAGG-3′)—SEQ ID NO:45 and the reverse primer HC2R (5′-TTATATTAAAAATGCCAAACCCTCCAG-3′)—SEQ ID NO:48 using the ligation reaction between FrNT and FrH23P as template. The fragment coding for the C-terminal domain (FrCT) is amplified using the forward primer CTD (5′-TGGATTTTTAAGTACGCAACGGGAGAGC-3′)—SEQ ID NO:49 containing MseI restriction site (underlined) and the reverse primer CTR (5′-AGCCATACTAGTAGTGTGTTGACCACCACGAG-3′)—SEQ ID NO:50 containing the SpeI restriction site (underlined) using Oleo-FL cDNA as template (FIG. 9c).

These PCR fragments are purified and digested with the enzyme MseI creating a cohesive ending in the 3′ end of the fragment FrNTH23P and in the 5′ end of the fragments FrCT. The fragments are purified from the digestion reaction and are then ligated together.

The modified oleosin OleoH23P (SEQ ID NO:3) is amplified using the forward primer NTD2 (5′-TATTCTCGAGCCATGGCGGATACTGCTAGAGG-3′)—SEQ ID NO:45 and the reverse primer CTR (5′-AGCCATACTAGTAGTGTGTTGACCACCACGAG-3′)—SEQ ID NO:50 using the ligation reaction between FrCT and FrNTH23P as template. (FIG. 9d).

Construction of OleoH3P

The fragment coding for the N-terminal domain (FrNT) is amplified using the forward primer NTD2 (5′-TATTCTCGAGCCATGGCGGATACTGCTAGAGG-3′)—SEQ ID NO:45 containing XhoI and NcoI restriction sites (underlined) and the reverse primer NTR (5′-CAGTGGCGCCTTTAGCAATCTGTCTAGAC-3′)—SEQ ID NO:46 containing the NarI restriction site (underlined) using Oleo-FL cDNA as template. The fragment FrH3P is amplified using the forward primer HN3D (5′-CCTTAGCGGCGCCGGAACTGTCATAGCTTTG-3′)—SEQ ID NO:51 containing NarI restriction site (underlined) and the reverse primer HC3R (5′-AGAGTTAAAAATACCGGTGATGAGGAGT-3′)—SEQ ID NO:52 containing the MseI restriction site (underlined) using Oleo-FL cDNA as template. (FIG. 9b).

These PCR fragments are purified and digested with the enzyme NarI creating cohesive ending in the 3′ end of the fragment FrNT and in the 5′ end of the FrH3P fragment. The fragments are purified from the digestion reaction and ligated together.

The fragment FrNTH3P is amplified using the forward primer NTD2 (5′-TATTCTCGAGCCATGGCGGATACTGCTAGAGG-3′)—SEQ ID NO:45 and the reverse primer HC3R (5′-AGAGTTAAAAATACCGGTGATGAGGAGT-3′)—SEQ ID NO:52 using the ligation reaction between FrNT and FrH3P as template. The fragment coding for the C-terminal domain (FrCT) is amplified using the forward primer CTD (5′-TGGATTTTTAAGTACGCAACGGGAGAGC-3′)—SEQ ID NO:49 containing MseI restriction site (underlined) and the reverse primer CTR (5′-AGCCATACTAGTAGTGTGTTGACCACCACGAG-3′)—SEQ ID NO:50 containing the SpeI restriction site (underlined) using Oleo-FL cDNA as template (FIG. 9c).

These PCR fragments are purified and digested with the enzyme MseI creating a cohesive ending in the 3′ end of the FrNTH3P and in the 5′ end of the FrCT fragment. The fragments are purified from the digestion reaction and ligated together.

The modified oleosin OleoH3P (SEQ ID NO:5) is amplified using the forward primer NTD2 (5′-TATTCTCGAGCCATGGCGGATACTGCTAGAGG-3′)—SEQ ID NO:45 and the reverse primer CTR (5′-AGCCATACTAGTAGTGTGTTGACCACCACGAG-3′)—SEQ ID NO:50 using the ligation reaction between FrCT and FrNTH3P as template (FIG. 9d).

Construction of OleoP

The fragment coding for the N-terminal domain (FrNT) is amplified using the forward primer NTD2 (5′-TATTCTCGAGCCATGGCGGATACTGCTAGAGG-3′)—SEQ ID NO:45 containing XhoI and NcoI restriction sites (underlined) and the reverse primer NTR (5′-CAGTGGCGCTTTAGCAATCTGTCTAGAC-3′)—SEQ ID NO:46 containing the NarI restriction site (underlined) using Oleo-FL cDNA as template. The fragment FrP is amplified using the forward primer PKMD (5′-ACTGTTGGCGCCCCTCTGCTCGTTATCTTC-3′)—SEQ ID NO:53 containing NarI restriction site (underlined) and the reverse primer PKMR (5′-TGTGTTAAAAATCGGGACAAGGATGGGGC-3′)—SEQ ID NO:54 containing the MseI restriction site (underlined) using Oleo-FL cDNA as template (FIG. 9b).

These PCR fragments are purified and digested with the enzyme NarI creating cohesive ending in the 3′ end of the fragment FrNT and in the 5′ end of the FrP fragment. The fragments are purified from the digestion reaction and ligated together.

The fragment FrNTP is amplified using the forward primer NTD2 (5′-TATTCTCGAGCCATGGCGGATACTGCTAGAGG-3′)—SEQ ID NO:45 and the reverse primer PKMR (5′-TGTGTTAAAAATCGGGACAAGGATGGGGC-3′)—SEQ ID NO:54 using the ligation reaction between FrNT and FrP as template. The fragment coding for the C-terminal domain (FrCT) is amplified using the forward primer CTD (5′-TGGATTTTTAAGTACGCAACGGGAGAGC-3′)—SEQ ID NO:49 containing MseI restriction site (underlined) and the reverse primer CTR (5′-AGCCATACTAGTAGTGTGTTGACCACCACGAG-3′)—SEQ ID NO:50 containing the SpeI restriction site (underlined) using Oleo-FL cDNA as template (FIG. 9c).

These PCR fragments are purified and digested with the enzyme MseI creating a cohesive ending in the 3′ end of the FrNTP fragment and in the 5′ end of the FrCT fragment. The fragments are purified from the digestion reaction and ligated together.

The modified oleosin OleoP (SEQ ID NO:7) is amplified using the forward primer NTD2 (5′-TATTCTCGAGCCATGGCGGATACIGCIAGAGG-3′)—SEQ ID NO:45 and the reverse primer CTR (5′-AGCCATACTAGTAGTGTGTTGACCACCACGAG-3′)—SEQ ID NO:50 using the ligation reaction between FrCT and FrNTP as template (FIG. 9d).

### Example 2

Reduction of the Hydrophobic Domain in Regions Flanking the Proline Knot Motif.

The hydrophobic domain from Oleo-FL can be reduced in the regions flanking the N- and C-terminal domain as described in FIG. 8c (Reduction in “direction 2”).

Construction of OleoH12P

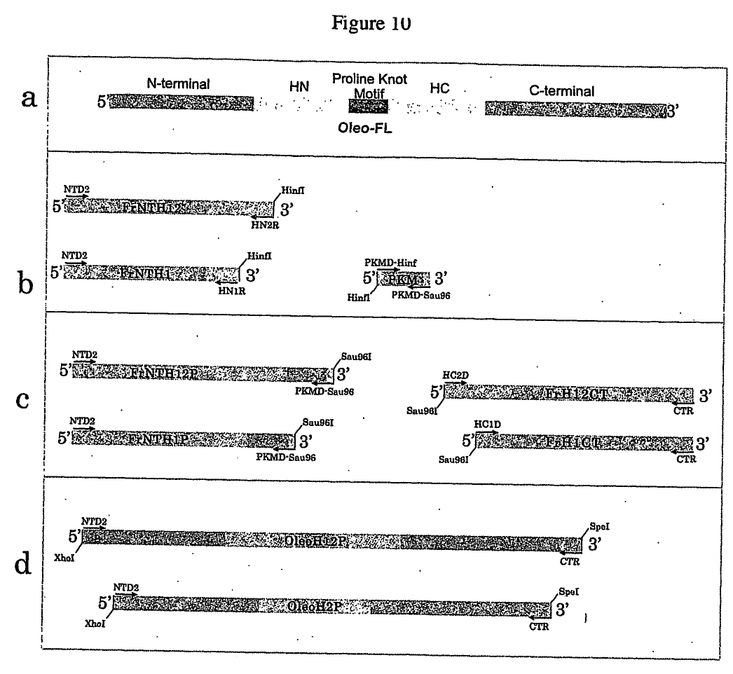

The fragment coding for the N-terminal domain plus regions I and II of the hydrophobic chain HN (FrNTH12) is amplified using the forward primer NTD2 (5′-TATTCTCGAGCCATGGCGGATACTGCTAGAGG-3′)—SEQ ID NO:45 containing XhoI and NcoI restriction sites (underlined) and the reverse primer HN2R (5′-ATAGGAGTCGCAACAAGGGTAAGGCTGGAGAG-3′)—SEQ ID NO:55 containing the HinfI restriction site (underlined) using Oleo-FL cDNA as template. The fragment coding for the proline knot motif (PKM) is amplified using the forward primer PKMD-Hinf (5′-TTGTGACTCCTCTTCTCGTTATCTTCAGCCCA-3′)—SEQ ID NO:56 containing HinfI restriction site (underlined) and the reverse primer PKMR-Sau96I (5′-ATGAGGGCCGGGACAAGGATTGGACTGAAGATAA-3′)—SEQ ID NO:57 containing the Sau961 restriction site (underlined) using Oleo-FL cDNA as template (FIG. 10b).

These PCR fragments are purified and digested with the enzyme HinfI creating cohesive ending in the 3′ end of the FrNTH12 fragment and in the 5′ end of the PKM fragment. The fragments are purified from the digestion reaction and ligated.

The fragment FrNTH12P is amplified using the forward primer NTD2 (5′-TATTCTCGAGCCATGGCGGATACTGCTAGAGG-3′)—SEQ ID NO:45 and the reverse primer PKMD-Sau96I (5′-ATGAGGGCCGGGACAAGGATTGGACTGAAGATAA-3′)—SEQ ID NO:57 using the ligation reaction between FrNTH12 and PKM as template. The fragment coding for the C-terminal domain plus regions I and II of the hydrophobic chain HC (FrCTH12) is amplified using the forward primer HC2D (5′-TCATGGCCCTATTTCTTTCCTCTGGAGG-3′)—SEQ ID NO:58 containing Sau96I restriction site (underlined) and the reverse primer CTR (5′-AGCCATACTAGTAGTGTGTTGACCACCACGAG-3′)—SEQ ID NO:50 containing the SpeI restriction site (underlined) using Oleo-FL cDNA as template (FIG. 10c).

These PCR fragments are purified and digested with the enzyme Sau96I creating a cohesive ending in the 3′ end of the FrNTH12P fragment and in the 5′ end of the FrH12CT fragment. The fragments are purified from the digestion reaction and ligated together.

The modified oleosin OleoH12P is amplified using the forward primer NTD2 (5′-TATTCTCGAGCCATGGCGGATACTGCTAGAGG-3′)—SEQ ID NO:45 and the reverse primer CTR (5′-AGCCATACTAGTAGTGTGTTGACCACCACGAG-3′)—SEQ ID NO:50 using the ligation reaction between FrNTH12P and FrNTH12P as template (FIG. 10d).

Construction of OleoH1P

The fragment coding for the N-terminal domain plus region I of the hydrophobic chain HN (FrNTH1) is amplified using the forward primer NTD2 (5′-TATTCTCGAGCCATGGCGGATACTGCTAGAGG-3′)—SEQ ID NO:45 and the reverse primer HN1R (5′-ATAGGAGTCGCGAGGGAACCACCAGCTGTG-3′)—SEQ ID NO:59 containing the HinfI restriction site (underlined) using Oleo-FL cDNA as template. The fragment coding for the proline knot motif (PKM) is amplified using the forward primer PKMD-Hinf (5′-TTGTGACTCCTCTCTCGTTATCTTCAGCCCA-3′)—SEQ ID NO:56 containing HinfI restriction site (underlined) and the reverse primer PKMR-Sau961 (5′-ATGAGGGCCGGGACAAGGATTGGACTGAAGATAA-3′)—SEQ ID NO:57 containing the Sau96I restriction site (underlined) using Oleo-FL cDNA as template (FIG. 10b).

These PCR fragments are purified and digested with the enzyme HinfI creating cohesive ending in the 3′ end of the FrNTH2 fragment and in the 5′ end of the fragment PKM. The fragments are purified from the digestion reaction and ligated together.

The fragment FrNTH1P is amplified using the forward primer NTD2 (5′-TATTCTCGAGCCATGGCGGATACTGCTAGAGG-3′)—SEQ ID NO:45 and the reverse primer PKMD-Sau96I (5′-ATGAGGGCCGGGACAAGGATTGGACTGAAGATAA-3′)—SEQ ID NO:57 using the ligation reaction between FrNTH1 and PKM as template. The fragment coding for the C-terminal domain plus region I of the hydrophobic chain HC (FrCTH1) is amplified using the forward primer HC1D (5′-GAGAGGCCCTAATTGCAGCTATAACCGTTTTC-3′)—SEQ ID NO:60 containing Sau961 restriction site (underlined) and the reverse primer CTR (5′-AGCCATACTAGTAGTGTGTTGACCACCACGAG-3′)—SEQ ID NO:50 containing the SpeI restriction site (underlined) using Oleo-FL cDNA as template (FIG. 10c).

These PCR fragments are purified and digested with the enzyme Sau96I creating a cohesive ending in the 3′ end of the FrNTH1P fragment and in the 5′ end of the FrH1CT fragment. The fragments are purified from the digestion reaction and ligated together.

The modified oleosin OleoH1P (SEQ ID NO:11) is amplified using the forward primer NTD2 (5′-TATTCTCGAGCCATGGCGGATACTGCTAGAGG-3′)—SEQ ID NO:45 and the reverse primer CTR (5′-AGCCATACTAGTAGTGTGTTGACCACCACGAG-3′)—SEQ ID NO:50 using the ligation reaction between FrNTH1P and FrCTH1P as template (FIG. 10d).

### Example 3

Enlargement of the Hydrophobic Domain.

Construction of Oleodouble

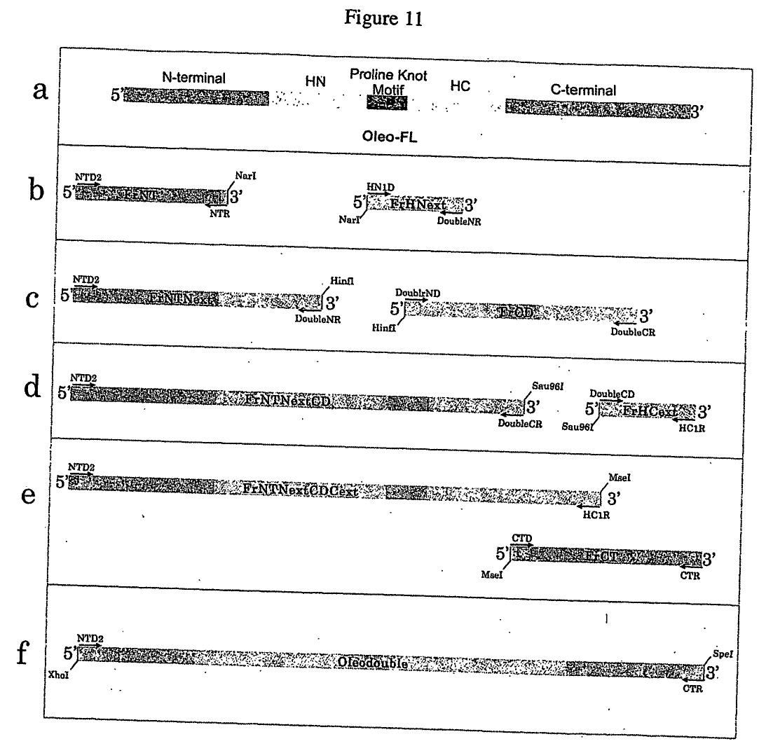

The hydrophobic domain from Oleo-FL can be extended. The fragment coding for the N-terminal domain (FrNT) is amplified using the forward primer NTD2 (5′-TATTCTCGAGCCATGGCGGATACTGCTAGAGG-3′)—SEQ ID NO:45 containing XhoI and NcoI restriction sites (underlined) and the reverse primer NTR (5′-CAGTGGCGCCTTTAGCAATCTGTCTAGAC-3′)—SEQ ID NO:46 containing the NarI restriction site (underlined) using Oleo-FL cDNA as template. The fragment coding for the extension of the HN hydrophobic chain (FrHNext) is amplified using the forward primer HN1D (5′-CTAAAGGCGCCACTGCTGTCACTGCTG-3′)—SEQ ID NO:61 containing NarI restriction site (underlined) and the reverse primer DoubleNR (5′-AGAGGAGTCACAACAGTCAAAGCTATCACAG-3′)—SEQ ID NO:62 containing the HinfI restriction site (underlined) using Oleo-FL cDNA as template (FIG. 11b).

The PCR fragments are purified and digested with the enzyme NarI creating cohesive ending in the 3′ end of the fragment FrNT and in the 5′ end of the fragment FrHNext. The fragments are purified from the digestion reaction and then ligated. The fragment FrNTNext is amplified using the forward primer NTD2 (5′-TATTCTCGAGCCATGGCGGATACTGCTAGAGG-3′)—SEQ ID NO:45 and and the reverse primer DoubleNR (5′-AGAGGAGTCACAACAGTCAAAGCTATCACAG-3′)—SEQ ID NO:62) using the previous ligation reaction as template. The fragment coding for the hydrophobic domain of Oleo-FL (FrCD) is amplified using the forward primer DoubleND (5′-AAACGACTCCTGCGGTCACAGCFAGTGGTTC-3′)—SEQ ID NO:63 containing HinfI restriction site (underlined) and the reverse primer DoubleCR (5′-TACAGGGCCATCCAAGAGAAAACACTTATAG-3′)—SEQ ID NO:64 containing the Sau96I restriction site (underlined) using Oleo-FL cDNA as template (FIG. 11c).

The PCR fragments are purified and digested with the enzyme HinfI creating cohesive ending in the 3′ end of the fragment FrNTNext and in the 5′ end of the fragment FrCD. The fragments are purified from the digestion reaction and then ligated. The fragment FrNTNextCD is amplified using the forward primer NTD2 (5′-TATTCTCGAGCCATGGCGGATACTGCTAGAGG-3′)—SEQ ID NO:45 and the reverse primer DoubleCR (5′-TACAGGGCCATCCAAGAGAAAACACTTATAG-3′)—SEQ ID NO:64 using the previous ligation reaction as template. The fragment coding for the extension of the HC hydrophobic chain (FrHCext) is amplified using the forward primer DoubleCD (5′-TCCCGGCCCTCATCACAGTTGCACTCCTC-3′)—SEQ ID NO:65 containing Sau96I restriction site (underlined) and the reverse primer HC1R (5′-GCGTAGTTAAAAATCCAAGAGAAAACGG-3′)—SEQ ID NO:66 containing the MseI restriction site (underlined) using Oleo-FL cDNA as template (FIG. 11d).

The PCR fragments are purified and digested with the enzyme Sau96I creating cohesive ending in the 3′ end of the fragment FrNTNextCD and in the 5′ end of the fragment FrHCext. The fragments are purified from the digestion reaction and then ligated. The fragment FrNTNextCDCext is amplified using the forward primer NTD2 (5′-TATTCTCGAGCCATGGCGGATACTGCTAGAGG-3′)—SEQ ID NO:45 and the reverse primer HC1R (5′-GCGTAGTTAAAAATCCAAGAGAAAACGG-3′)—SEQ ID NO:66 using the previous ligation reaction as template. The fragment coding for the C-terminal domain (FrCT) is amplified using the forward primer CTD (5′-TGGATTAAGTACGCAACGGGAGAGC-3′)—SEQ ID NO:49 containing MseI restriction site (underlined) and the reverse primer CTR (5′-AGCCATACTAGTAGTGTGTTGACCACCACGAG-3′)—SEQ ID NO:50 containing the SpeI restriction site (underlined) using Oleo-FL cDNA as template (FIG. 11e).

The PCR fragments are purified and digested with the enzyme MseI creating cohesive ending in the 3′ end of the fragment FrNTNextCD and in the 5′ end of the fragment FrHCext. The fragments are purified from the digestion reaction and then ligated. The modified oleosin Oleo-double is amplified using the forward primer NTD2 (5′-TATTCTCGAGCCATGGCGGATACTGCTAGAGG-3′)—SEQ ID NO:45 and the reverse primer CTR (5′-AGCCATACTAGTAGTGTGTTGACCACCACGAG-3′)—SEQ ID NO:50 using the previous ligation reaction as template (FIG. 11f).

### Example 4

The Use of Modified Oleosins as Carriers for Recombinant Proteins (Green Fluorescent Protein) in Plants.

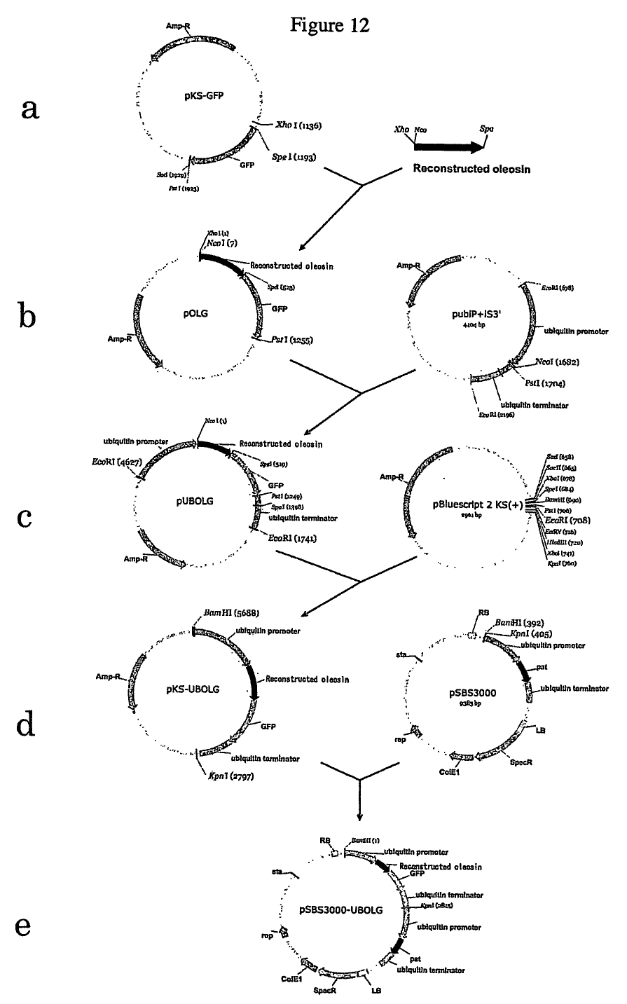

The recombinant green fluorescent protein is amplified using the forward primer GFPdir (5′-TTACTACTAGTATGGCATCTAAAGGAGAAGAACT-3′)—SEQ ID NO:67 containing the SpeI restriction site (underlined) and the reverse primer GFPrev (5′-ATCGGAGCTCCTGCAGTTATTTGTATAGTTCATCCATGCC-3′)—SEQ ID NO:68 containing the PstI restriction site (underlined) using GFP cDNA as template. The PCR fragment is purified and digested with the enzymes SpeI and PstI creating cohesive endings in both 3′ and 5′ ends. The fragment is purified from the digestion reaction and then ligated to pBluescript KS+ (Stratagene) previously digested with SpeI and PstI. The product of this ligation is called pKS-GFP (FIG. 12a).

The modified oleosins (OleoH12P, OleoH23P, OleoH1P, OleoH3P, OleoP and OleoDouble) as well as a non-modified Oleo-FL, amplified with the Forward primer NTD2 (5′-TATTCTCGAGCCATGGCGGATACIGCIAGAGG-3′)—SEQ ID NO:45 and the reverse primer CTR (5′-AGCCATACTAGTAGTGTGTTGACCACCACGAG-3′)—SEQ ID NO:50 using Oleo-FL cDNA as template are digested with XhoI and SpeI. The fragments are purified and inserted in the vector pKS-GFP previously digested with XhoI and SpeI. During amplification procedures the reverse primer CTR removes the stop codon from the oleosin and adds a SpeI site to assist in creating an in-frame translation fusion with GFP. The vectors obtained with ligation of Oleo-FL, OleoH12P, OleoH23P, OleoH1P, OleoH3P, OleoP and OleoDouble are called pOLG-FL, pOLG-OleoH12P, pOLG-OleoH23P, pOLG-OleoH1P, pOLG-OleoH3P, pOLG-OleoP and pOLG-OleoDouble. To summarize they are simply called pOLG in FIG. 12b.

The “open reading frame” fusions oleosin-GFP obtained in the pOLG series plasmids are digested with NcoI and PstI. The fragments are individually inserted into the vector pubiP+IS3′ previously digested with the same enzymes (FIG. 12b) creating the plasmids pUBOL-GFL, pUBOLG-OleoH12P, pUBOLG-OleoH23P, pUBOLG-OleoH1P, pUBOLG-OleoH3P, pUBOLG-OleoP and pUBOLG-OleoDouble. To summarize they are simply called pUBOLG in FIG. 12c. The cloning strategy in this step is designed to insert the fusions between the parsley ubiquitin promoter and terminator (Kawalleck, P. et al., 1993 Plant Mol. Biol. 21: 673-684). The first transcribed “ATG” is the first methionine codon from oleosin.

The cassettes containing ubiquitin ptomoter followed by oleosin-GFP fusions followed by ubiquitin terminator are digested with EcoRI. The fragments are individually subcloned into the vector pBluescript KS+previously digested with EcoRI (FIG. 12c). The plasmids created are named pKSUBOLG-FL, pKSUBOLG-OleoH12P, pKSUBOLG-OleoH23P, pKSUBOLG-OleoH1P, pKSUBOLG-OleoH3P, pKSUBOLG-OleoP and pKSUBOLG-OleoDouble (named as pKSUBOLG in FIG. 12d).

The cassettes in the pKSUBOLG plasmids are then removed from pBluescript backbone using digestion with BamHI and KpnI restriction enzymes and individually inserted in the binary vector pSBS3000 previously digested with the same enzymes (FIG. 12d). The binary vectors created are named pSBS3000-UBOLG-FL, pSBS3000-UBOLG-OleoH12P, pSBS3000-UBOLG-OleoH23P, pSBS3000-UBOLG-OleoH1P, pSBS3000-UBOLG-OleoH3P, pSBS3000-UBOLG-OleoP and pSBS3000-UBOLG-OleoDouble (named as pSBS3000-UBOLG in FIG. 12e). The pSBS3000 vector contains the pat cassette composed of phosphinothricine acetyl transferase cDNA driven by the parsley ubiquitin promoter and terminator. This cassette confers host plant resistance to the herbicide phosphinothricine (Wohlleben, W. et al., 1988 Gene 70: 25-37). The pat and oleosin cassettes are present between two regions called right and left border (RB and LB respectively). The DNA fragment between these two regions is called T-DNA and can be inserted into a plant genome via Agrobacterium transformation.

### Example 5

Agrobacterium and Arabidopsis Transformation

The binary vectors pSBS3000-UBOLG-FL, pSBS3000-UBOLG-OleoH12P, pSBS3000-UBOLG-OleoH23P, pSBS3000-UBOLG-OleoH1P, pSBS3000-UBOLG-OleoH3P, pSBS3000-UBOLG-OleoP and pSBS3000-UBOLG-OleoDouble are individually transformed into Agrobacterium EHA101 (Hood, E. E. et al., 1986 J. Bacteriol. 168: 1291-1301) by electroporation method. The transformed Agrobacterium lines containing the binary vector are selected spectinomycin resistance (“SpecR” in FIG. 12e). One line of Agrobacterium is selected for each construct.

Arabidopsis thaliana ecotype C24 is used for transformation. Five seeds are planted on the surface of a soil mixture (two-thirds Redi-earth and one-third perlite with a pH=6.7) in 4 inch pots. The seedlings are allowed to grow to a rosette stage of 6-8 leaves to a diameter of approximately 2.5 cm. These seedlings are transplanted into 4 inch pots containing the above soil mixture, covered with window screen material which has five 1 cm diameter holes cut into the mesh; one in each of the corners, and one in the center. The pots are placed inside a dome at 4° C. for four days for a cold treatment and subsequently moved to 24° C. growth room with constant light at about 150 μE and 50% relative humidity. The plants are irrigated at 2-3 day interval and fertilized weekly with 1% of Peters 20-20-20. When stems reach about 2 cm in height, the primary bolts are cut to encourage the growth of secondary and tertiary bolts. Four to five days after cutting the primary bolts, the plants are ready to be infected with Agrobacterium.

The Agrobacterium lines are individually inoculated in 500 ml of LB media and grown until reach optical density of 0.8 at 600 nm. The cultures are centrifuged precipitate the bacteria that is suspended in a solution containing 5% of sucrose and 0.05% of the surfactant Silwet L-77 (Lehle Seeds).

The pots with Arabidopsis plants are inverted this solution for 20 seconds. The pots are subsequently covered with a transparent plastic dome for 24 hours to maintain higher humidity. The plants are allowed to grow to maturity and seeds (untransformed and transformed) are harvested.

For selection of transgenic lines, the putative transformed seeds are sterilized with a quick wash of 70% ethanol and a treatment in 20% commercial bleach for 15 min. The bleach solution is removed by rinsing seeds four times with water. About 1000 sterilized seeds are mixed with 0.6% top agar and evenly spread on a half strength MS plate (Murashige and Skoog, 1962 Physiologia Plantarium 15: 473-497) containing 1% sucrose and 80 μM of the herbicide phosphinothricin (PPT) DL. The plates are then placed in a growth room with light regime 8 hr dark and 16 hr light at 24° C. After 7 to 10 days, putative transgenic seedlings are green and growing whereas untransformed seedlings die. After the establishment of roots the putative transgenic seedlings are individually transferred to pots (the individually plants are irrigated in 3 day interval and fertilized with 1% Peters 20-20-20 in 5 day interval and allowed to grow to maturity. The pots are covered with a transparent plastic dome for three days to protect the sensitive seedlings. After 7 days the seedlings are covered with a seed collector from Lehle Seeds to prevent seed loss due to scattering. Seeds from these transgenic plants are harvested individually and ready for analysis.

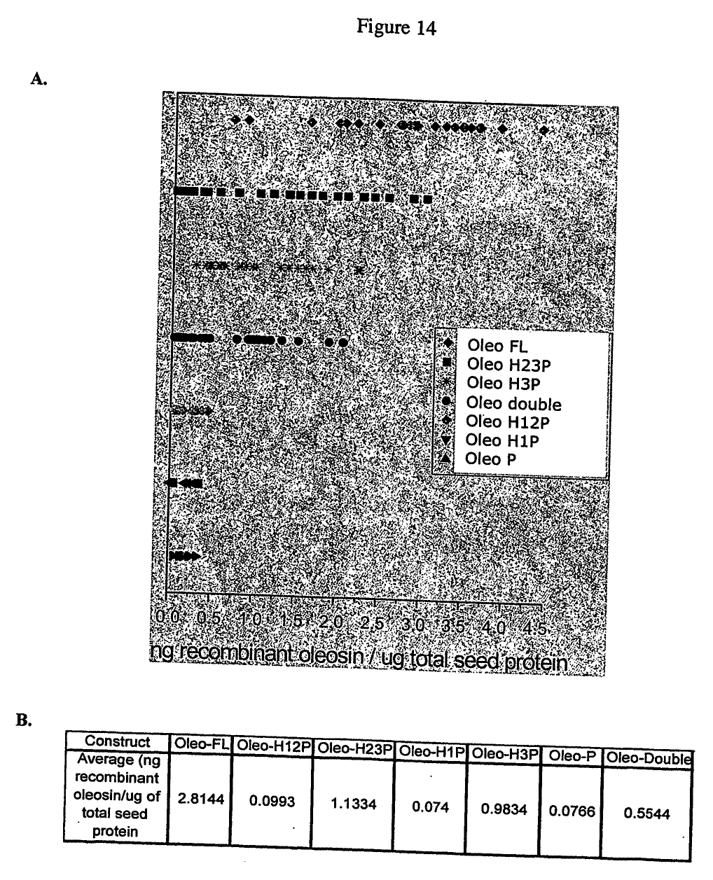

Ten milligrams of seeds from each transgenic plant are grinded in total protein extraction buffer (2% SDS, 5 mM EDTA, 50 mM Tris-Cl, pH 6.8). The extracts are incubated in boiling water for 5 minutes and centrifuged at full speed for 10 minutes. The soluble phase is removed and stored at −20° C. for further analysis. The presence of recombinant oleosin fused to GFP is analyzed by western blot using anti-GFP antibody. GFP standards are used in each experiment to allow comparison of different recombinant proteins. The intensity of each band in the membranes is measured using a densitometer. The amounts of each band are normalized with the GFP standards and plotted (FIG. 14). Seeds from plants showing the highest expression for each recombinant oleosin with exception of OleoFL are propagated for two more generations. In the case of OleoFL a plant showing intermediate accumulation is propagated.

### Example 6

Isolation of Oil Bodies

Seeds are recovered from the selected plants as previously described. The isolation of oil bodies from these seeds is performed using the method reported by van Rooijen & Moloney, (1995b), Biotechnol 13: 72-77 with the following modifications. Briefly, 50 mg of dry mature seeds are grinded inside a 1.7 ml microfuge tube with 1.0 ml of low stringency phosphate buffer (100 mM phosphate buffer pH 8 with 0.5M NaCl). The extract is centrifuged for 15 min at 10,000 g at room temperature (RT). After centrifugation the fat pad containing the oil bodies is removed from the aqueous phase and transferred to another microfuge tube. The oil bodies are resuspended in 1.0 ml of low stringency phosphate buffer (100 mM phosphate buffer pH 8 with 0.5M NaCl). The sample is centrifuged for 15 minutes at 10,000 g at RT and the undertatant is removed. The oil bodies are subsequently resuspended in a 0.5 ml of high stringency urea buffer (8M Urea in 100 mM Na-Carbonate buffer pH 8.0). The sample is centrifuged for 15 min at 10,000 g at 4° C. and the undernatant removed. The oil bodies are finally suspended in 0.3 ml of water.

### Example 7

Confocal Microscopy for Modified Oleosin Localization

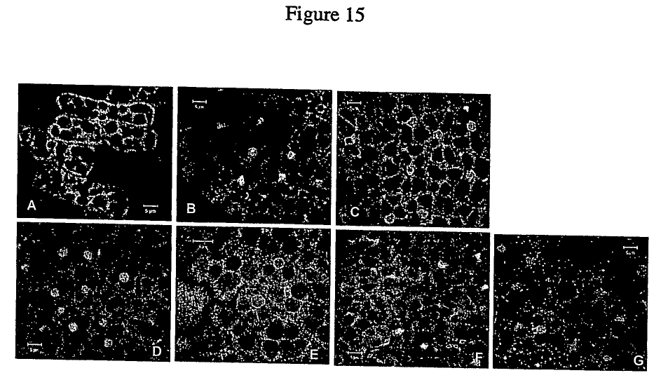

Mature embryos are isolated according the method established by Perry and Wang (2003) Biotechniques 35:278-281. Isolated embryos are examined with a Zeiss LSM 510 laser scanning confocal microscope using line-sequential single-tracking mode with the AOTF-controlled excitation with 488 nm and 543 nm laser (set at 20% and 100% respectively). A Plan-Appochromat 40×/1.4 Oil DIC objective is used with scan zoom. The pinhole is optimized for about 100 μm. The resulting micrographs is shown in FIG. 15. Oil bodies are present in the boundaries of the cells as it is shown for the non-modified recombinant oleosin Oleo-FL (FIG. 15a). The modified oleosins are also present in the boundaries of the cells in small hollow structures that are oil bodies.

### Example 8

Differential Affinities of Modified Oleosins to Oil Bodies

Fifty milligrams of Arabidopsis transgenic seeds carrying the Oleo-FL or the modified oleosins (Oleo-H12P, Oleo-H23P, Oleo-H1P, Oleo-H3P, Oleo-P or Oleo-double, each one attached to GFP) were grinded in 1.5 ml of TrisHCl 100 mM pH 8.0 with mortar and pestle. The sample was centrifuged at 13,000 rpm for 15 min. The aqueous phase was recovered with a needle and syringe. The fat pad was suspended in 300 μl of TrisHCl pH 8.0 100 mM and centrifuged at 13,000 rpm for 10 minutes in a 0.5 ml eppendorf tube. The aqueous phase was recovered and the fat pad was suspended in 50 μl of sodium chloride 1M. The suspension was incubated at room temperature for 10 minutes and centrifuged at 13,000 rpm for 10 minutes. The aqueous phase was recovered and the fat pad was suspended in 50 μl of tween 20 5%. The suspension was incubated at room temperature for 10 minutes and centrifuged at 13,000 rpm for 10 minutes. The aqueous phase was recovered and the fat pad was suspended in 50 μl of CHAPS 5%. The suspension was incubated at room temperature for 10 minutes and centrifuged at 13000 rpm for 10 minutes. The aqueous phase was recovered and the fat pad was suspended in 50 μl of sodium carbonate 100 mM pH 10.0. The suspension was incubated at room temperature for 10 minutes and centrifuged at 13,000 rpm for 10 minutes. The aqueous fraction was recovered and the fat pad was suspended in 50 μl of urea 9M. The suspension was incubated at room temperature for 10 minutes and centrifuged at 13000 rpm for 10 minutes. The aqueous phase was recovered and the fat pad was suspended in 50 μl of SDS 5%. The suspension was incubated at room temperature for 10 minutes and centrifuged at 13,000 rpm for 10 minutes. The aqueous phase was recovered. The fat pad was suspended in 75 μl of SDS-PAGE loading buffer. The 50 μl aqueous phase fractions were mixed with 25 μl of SDS-PAGE loading buffer (3× concentrated). The samples were incubated in a water boiling bath for 5 minutes and loaded in SDS-PAGE 10%. After electrophoretical separation the proteins were transferred to PVDF membranes. The recombinant proteins were developed through western blot using an anti-GFP antibody.

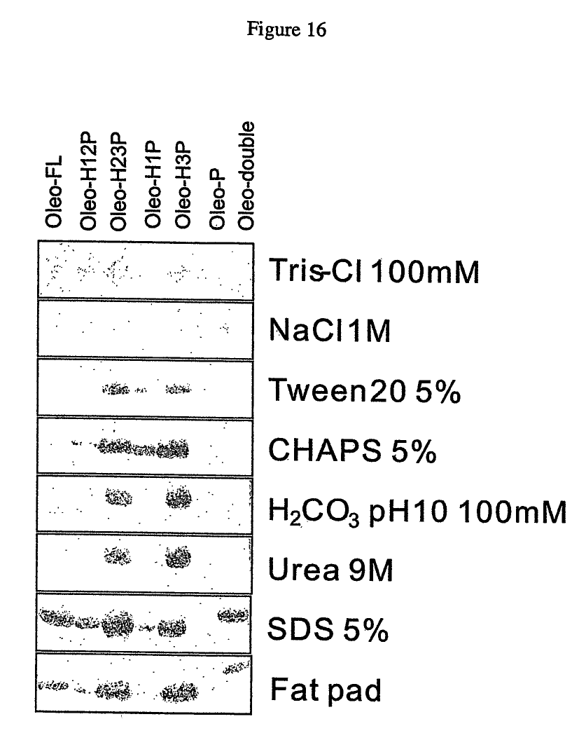

As shown in FIG. 16, the modified oleosins display differential affinity of modified oleosins to oil bodies compared to the non-modified oleosin (Oleo-FL). The modified oleosins containing shorter hydrophobic domains (Oleo H12P, OleoH23P, OleoH1P and Oleo H3P) could be selectively extracted from the oil bodies using some detergent solutions, like the zwitterionic detergent CHAPS (3-[(3-cholamidopropyl)dimethylammonio]-1-propanesulfonate) and the non-ionic detergent Tween20 (polyoxyethylenesorbitan monooleate) at concentration of 5% (v/v). Other detergents, such as the anionic detergent SDS (sodium dodecyl sulfate) at concentration of 5% (w/v) displayed no selectivity because it extracted the modified and non-modified oleosins. Non-detergent solutions like sodium carbonate 100 mM, pH 10.0 and urea 9M also displayed selectivity for the extraction of modified oleosins containing shorter hydrophobic domains.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### SUMMARY OF SEQUENCES

SEQ ID NO:1 and 2 set forth the nucleotide sequence and the deduced amino acid sequence, respectively, of the Oleo-FL clone.

SEQ ID NO:3 and 4 set forth the nucleotide sequence and the deduced amino acid sequence, respectively, of the Oleo-H23P clone.

SEQ ID NO:5 and 6 set forth the nucleotide sequence and the deduced amino acid sequence, respectively, of the Oleo-H3P clone.

SEQ ID NO:7 and 8 set forth the nucleotide sequence and the deduced amino acid sequence, respectively, of the Oleo-P clone.

SEQ ID NO:9 and 10 set forth the nucleotide sequence and the deduced amino acid sequence, respectively, of the Oleo-H12P clone.

SEQ ID NO:11 and 12 set forth the nucleotide sequence and the deduced amino acid sequence, respectively, of the Oleo-H1P clone.

SEQ ID NO:13 and 14 set forth the nucleotide sequence and the deduced amino acid sequence, respectively, of the Oleo-double clone.

SEQ ID NO:15 sets forth the amino acid sequence of the Arabidopsis thaliana oleosin protein SEQ ID NO:16 sets forth the amino acid sequence of the Brassica napus oleosin protein.

SEQ ID NO:17 and 18 set forth the nucleotide sequences of an Oleo-FL N-terminal domain.

SEQ ID NO:19 to 20 set forth the nucleotide sequences of an Oleo-FL Hydrophobic HN domain 1a.

SEQ ID NO:21 sets forth the nucleotide sequence of an Oleo-FL Hydrophobic HN domain 2a.

SEQ ID NO:22 sets forth the nucleotide sequence of an Oleo-FL Hydrophobic HN domain 3a.

SEQ ID NO:23 and 24 set forth the nucleotide sequence of an Oleo-FL proline knot.

SEQ ID NO: 25 and 26 set forth the nucleotide sequences of an Oleo-FL Hydrophobic HC domain 1b.

SEQ ID NO:27 sets forth the nucleotide sequence of an Oleo-FL Hydrophobic HC domain 2b.

SEQ ID NO:28 sets forth the nucleotide sequence of an Oleo-FL Hydrophobic HC domain 3b.

SEQ ID NO: 29 and 30 set forth the nucleotide sequences of an Oleo-FL C-terminal domain.

SEQ ID NO: 31 and 32 set forth the amino acid sequences of an Oleo-FL N-terminal domain.

SEQ ID NO: 33 to 34 set forth the amino acid sequences of an Oleo-FL Hydrophobic HN domain 1a.

SEQ ID NO:35 sets forth the amino acid sequence of an Oleo-FL Hydrophobic HN domain 2a.

SEQ ID NO:36 sets forth the amino acid sequence of an Oleo-FL Hydrophobic HN domain 3a.

SEQ ID NO:37 and 38 set forth the amino acid sequence of an Oleo-FL proline knot.

SEQ ID NO: 39 and 40 set forth the amino acid sequences of an Oleo-FL Hydrophobic HC domain 1b.

SEQ ID NO:41 sets forth the amino acid sequence of an Oleo-FL Hydrophobic HC domain 2b.

SEQ ID NO:42 sets forth the amino acid sequence of an Oleo-FL Hydrophobic HC domain 3b.

SEQ ID NO: 43 and 44 set forth the amino acid sequences of an Oleo-FL C-terminal domain.

SEQ ID NO:45 sets forth the nucleotide sequence of the forward primer NTD2 which is complementary to the 5′ region Arabidopsis Oleo-FL oleosin and is designed to add a XhoI and NcoI site to the 5′ region facilitate subsequent ligation.

SEQ ID NO:46 sets forth the nucleotide sequence of the reverse primer NTR which is complementary to the region junction between the N-terminal region of Oleo-FL and the HN hydrophobic domain and is designed to add a NarI site to the 3′ region facilitate subsequent ligation.

SEQ ID NO:47 sets forth the nucleotide sequence of the forward primer HN2D which is complementary to domain 2 of the HN hydrophobic domain of Oleo-FL oleosin and is designed to add a NarI site to the 5′ region facilitate subsequent ligation.

SEQ ID NO:48 sets forth the nucleotide sequence of the reverse primer HC2R which is complementary domain 2 of the HC hydrophobic domain of the Oleo-FL oleosin and is designed to add a MseI site to the 3′ region facilitate subsequent ligation.

SEQ ID NO:49 sets forth the nucleotide sequence of the forward primer CTD which is complementary to junction between the HC hydrophobic domain and the C-terminal domain of Oleo-FL oleosin and is designed to add a MseI site to the 5′ region facilitate subsequent ligation.

SEQ ID NO:50 sets forth the nucleotide sequence of the reverse primer CTR which is complementary to 3′ region of the C-terminal domain of Oleo-FL oleosin and is designed to add a SpeI site to the 3′ region facilitate subsequent ligation.

SEQ ID NO:51 sets forth the nucleotide sequence of the forward primer HN3D which is complementary to domain 3 of the HN hydrophobic domain of Oleo-FL oleosin and is designed to add a NarI site to the 5′ region facilitate subsequent ligation.

SEQ ID NO:52 sets forth the nucleotide sequence of the reverse primer HC3R which is complementary domain 3 of the HC hydrophobic domain of the Oleo-FL oleosin and is designed to add a MseI site to the 3′ region facilitate subsequent ligation.

SEQ ID NO:53 sets forth the nucleotide sequence of the forward primer PKMD which is complementary to junction between the HN hydrophobic domain and the proline knot motif of Oleo-FL oleosin and is designed to add a NarI site to the 5′ region facilitate subsequent ligation.

SEQ ID NO:54 sets forth the nucleotide sequence of the reverse primer PKMR which is complementary to junction between the proline knot and the HC hydrophobic domain of Oleo-FL oleosin and is designed to add a MseI site to the 3′ region facilitate subsequent ligation.

SEQ ID NO:55 sets forth the nucleotide sequence of the reverse primer HN2R which is complementary to domain 2 of the HN hydrophobic domain of Oleo-FL oleosin and is designed to add a HinfI site to the 3′ region facilitate subsequent ligation.

SEQ ID NO:56 sets forth the nucleotide sequence of the forward primer PKMD-Hinf which is complementary to junction between the HN hydrophobic domain and the proline knot motif of Oleo-FL oleosin and is designed to add a HinfI site to the 5′ region facilitate subsequent ligation.

SEQ ID NO:57 sets forth the nucleotide sequence of the reverse primer PKMR-Sau96I which is complementary to junction between the proline knot and the HC hydrophobic domain of Oleo-FL oleosin and is designed to add a Sau96I site to the 3′ region facilitate subsequent ligation.

SEQ ID NO:58 sets forth the nucleotide sequence of the forward primer HC2D which is complementary to domain 2 of the HC hydrophobic domain of Oleo-FL oleosin and is designed to add a Sau96I site to the 5′ region facilitate subsequent ligation.

SEQ ID NO:59 sets forth the nucleotide sequence of the reverse primer HN1R which is complementary to domain 1 of the HN hydrophobic domain of Oleo-FL oleosin and is designed to add a HinfI site to the 3′ region facilitate subsequent ligation. SEQ ID NO:60 sets forth the nucleotide sequence of the forward primer HC1D which is complementary to domain 1 of the HC hydrophobic domain of Oleo-FL oleosin and is designed to add a Sau96I site to the 5′ region facilitate subsequent ligation.

SEQ ID NO:61 sets forth the nucleotide sequence of the forward primer HN1D which is complementary to domain 1 of the HN hydrophobic domain of Oleo-FL oleosin and is designed to add a NarI site to the 5′ region facilitate subsequent ligation.

SEQ ID NO:62 sets forth the nucleotide sequence of the reverse primer DoubleNR which is complementary to 3′ region of the HN hydrophobic domain of Oleo-FL oleosin and is designed to add a HinfI site to the 3′ region facilitate subsequent ligation.

SEQ ID NO:63 sets forth the nucleotide sequence of the forward primer DoubleND which is complementary to 5′ region of the HN hydrophobic domain of Oleo-FL oleosin and is designed to add a HinfI site to the 5′ region facilitate subsequent ligation.

SEQ ID NO:64 sets forth the nucleotide sequence of the reverse primer DoubleCR which is complementary to 3′ region of the HC hydrophobic domain of Oleo-FL oleosin and is designed to add a Sau96I site to the 3′ region facilitate subsequent ligation.

SEQ ID NO:65 sets forth the nucleotide sequence of the forward primer DoubleCD which is complementary to 5′ region of the HC hydrophobic domain of Oleo-FL oleosin and is designed to add a Sau96I site to the 5′ region facilitate subsequent ligation.

SEQ ID NO:66 sets forth the nucleotide sequence of the reverse primer HC1R which is complementary to domain 1 of the HC hydrophobic domain of Oleo-FL oleosin and is designed to add a MseI site to the 3′ region facilitate subsequent ligation.

SEQ ID NO:67 sets forth the nucleotide sequence of the forward primer GFPder which is complementary to 5′ region GFP and is designed to add a SpeI site to the 5′ region facilitate subsequent ligation.

SEQ ID NO:68 sets forth the nucleotide sequence of the reverse primer GFPrev which is complementary to 3′ region GFP and is designed to add a PstI site to the 3′ region facilitate subsequent ligation.

1. A modified oleosin polypeptide comprising (a) a proline knot motif and (b) a hydrophobic domain wherein the hydrophobic domain is modified by the removal or addition of at least one amino acid residue on both the amino terminal (HN) and carboxy terminal (HC) chains of the hydrophobic domain.

2. A modified oleosin according to claim 1 wherein at least one amino acid residue is removed from both the HN and HC chains of the hydrophobic domain.

3. A modified oleosin according to claim 2 wherein at least 2 to 28 amino acid residues are removed from both the HN and HC chains of the hydrophobic domain.

4. A modified oleosin according to claim 1 wherein sub-domains 1a and 1b are removed.

5. A modified oleosin according to claim 1 wherein sub-domains 2a and 2b are removed.

6. A modified oleosin according to claim 1 wherein sub-domains 3a and 3b are removed.

7. A modified oleosin according to any one of claims 1 to 3 comprising sub-domains 1a and 1b.

8. A modified oleosin according to claim 7 wherein sub-domain 1a has the sequence shown in SEQ ID NO:33 or SEQ ID NO:34 and sub-domain 1b has the sequence shown in SEQ ID NO:39 or SEQ ID NO:40.

9. A modified oleosin according to any one of claims 1 to 3 comprising sub-domains 2a and 2b.

10. A modified oleosin according to claim 9 wherein sub-domain 2a has the sequence shown in SEQ ID NO:35 and sub-domain 2b has the sequence shown in SEQ ID NO:41.

11. A modified oleosin according to any one of claims 1 to 3 comprising sub-domains 3a and 3b.

12. A modified oleosin according to claim 11 wherein sub-domain 3a has the sequence shown in SEQ ID NO:36 and sub-domain 3b has the sequence shown in SEQ ID NO:42.

13. A modified oleosin according to claim 2 comprising the amino acid sequence shown in SEQ ID NO:4, SEQ ID NO:6, SEQ ID NO:8, SEQ ID NO:10 or SEQ ID NO:12 or a functional variant thereof.

14. A modified oleosin according to claim 1 wherein at least one amino acid residue is added to both the HN and HC chains of the hydrophobic domain.

15. A modified oleosin according to claim 14 wherein at least 2 to 28 amino acid residues are added to both the HN and HC chains of the hydrophobic domain.

16. A modified oleosin according to claim 14 wherein a second copy of sub-domains 1a and 1b are added.

17. A modified oleosin according to claim 14 wherein a second copy of sub-domains 2a and 2b are added.

18. A modified oleosin according to claim 14 wherein a second copy of sub-domains 3a and 3b are added.

19. A modified oleosin according to claim 14 wherein a second copy of sub-domains 1a, 1b, 2a and 2b are added.

20. A modified oleosin according to claim 14 wherein a second copy of sub-domains 2a, 2b, 3a and 3b are added.

21. A modified oleosin according to claim 14 wherein a second copy of sub-domains 1a, 1b, 3a and 3b are added.

22. A modified oleosin according to claim 14 wherein a second copy of sub-domains 1a, 1b, 2a, 2b, 3a and 3b are added.

23. A modified oleosin according to any one of claims 14-16, 19, 21 and 22 wherein sub-domain 1a has the sequence shown in SEQ ID NO:33 or SEQ ID NO:34 and sub-domain 1b has the sequence shown in SEQ ID NO:39 or SEQ ID NO:40.

24. A modified oleosin according to any one of claims 14, 15, 17, 19, 20 and 22 wherein sub-domain 2a has the sequence shown in SEQ ID NO:35 and sub-domain 2b has the sequence shown in SEQ ID NO:41.

25. A modified oleosin according to any one of claims 14, 15, 18 and 20-22 wherein sub-domain 3a has the sequence shown in SEQ ID NO:36 and sub-domain 3b has the sequence shown in SEQ ID NO:42.

26. A modified oleosin according to claim 14 comprising the amino acid sequence shown in SEQ ID NO:14 or a functional variant thereof.

27. A modified oleosin according to claim 1 wherein said proline knot motif is selected from the sequences SEQ ID NO:37 or SEQ ID NO:38.

28. A modified oleosin according to claim 1 further comprising a N-terminal domain.

29. A modified oleosin according to claim 28 wherein said N-terminal domain is selected from the sequences SEQ ID NO:31 or SEQ ID NO:32.

30. A modified oleosin according to claim 1 further comprising a C-terminal domain.

31. A modified oleosin according to claim 30 wherein said C-terminal domain is selected from the sequences SEQ ID NO:43 or SEQ ID NO:44.

32. A nucleic acid sequence encoding a modified oleosin polypeptide according to claim 1.

33. A nucleic acid sequence according to claim 32 having the sequence as shown in SEQ ID NO:3, SEQ ID NO:5, SEQ ID NO:7, SEQ ID NO:9, SEQ ID NO:11 or SEQ ID NO:13 or a functional variant thereof.

34. A recombinant fusion protein comprising a modified oleosin polypeptide according to claim 1 fused to a heterologous protein.

35. A chimeric nucleic acid sequence, capable of being expressed in association with an oil body of a host cell comprising:
1) a first nucleic acid sequence capable of regulating the transcription in said host cell;
2) a second nucleic acid sequence, wherein said second sequence encodes a fusion polypeptide and comprises (i) a nucleic acid sequence according to claim 32 or 33 to provide targeting of the fusion polypeptide to a lipid phase linked in reading frame to (ii) a nucleic acid sequence encoding the heterologous polypeptide; and
3) a third nucleic acid sequence encoding a termination region functional in the host cell.

36. A cell comprising a modified oleosin polypeptide according to claim 1.

37. A cell comprising a fusion protein according to claim 34.

38. A cell comprising a nucleic acid sequence according to claim 32 or 33.

39. A cell comprising a chimeric nucleic acid sequence according to claim 35.

40. A plant seed comprising a modified oleosin polypeptide according to claim 1.

41. A plant seed comprising a fusion protein according to claim 34.

42. A plant seed comprising a nucleic acid sequence according to claim 32 or 33.

43. A plant seed comprising a chimeric nucleic acid sequence according to claim 35.

44. A plant comprising a modified oleosin polypeptide according to claim 1.

45. A plant comprising a fusion protein according to claim 34.

46. A plant comprising a nucleic acid sequence according to claim 32 or 33.

47. A plant comprising a chimeric nucleic acid sequence according to claim 35.

48. A method for the production of a recombinant polypeptide in a cell said method comprising:
a) introducing into a host cell a chimeric nucleic acid sequence comprising:
1) a first nucleic acid sequence capable of regulating the transcription in said host cell;
2) a second nucleic acid sequence, wherein said second sequence encodes a fusion polypeptide and comprises (i) a nucleic acid sequence encoding a sufficient portion of a modified oleosin protein according to claim 1 to provide targeting of the fusion polypeptide to a lipid phase linked in reading frame to (ii) a nucleic acid sequence encoding the recombinant polypeptide; and
3) a third nucleic acid sequence encoding a termination region functional in the host cell; and
b) growing said host cell to produce the fusion polypeptide.

49. The method according to claim 48 wherein said host cell is a plant cell.

50. The method according to claim 48 wherein said plant is dicotyledonous.

51. The method according to claim 48 wherein said plant is monocotyledonous.

52. The method according to claim 51 wherein said plant is from the species rapeseed (Brassica spp.), linseed/flax (Linum usitatissimum), safflower (Carthamus tinctorius), sunflower (Helianthus annuus), maize (Zea mays), soybean (Glycine max), mustard (Brassica spp. and Sinapis alba), crambe, (Crambe abyssinica), eruca (Eruca sativa), oil palm (Elaeis guineeis), cottonseed (Gossypium spp.), groundnut (Arachis hypogaea), coconut (Cocus nucifera), castor bean (Ricinus communes), coriander (Coriandrum sativum), squash, (Cucurbita maxima), Brazil nut (Bertholletia excelsa) and jojoba (Simmondsia chinensis).

53. A method according to claim 48 wherein said modified oleosin protein is selected from a group consisting of SEQ ID NO:4, SEQ ID NO:6, SEQ ID NO:8, SEQ ID NO:10, SEQ ID NO:12 or SEQ ID NO:14 or a functional variant thereof.


SEQUENCE LISTING (68 sequences)

SEQ ID NO: 1 - DNA, 519 bp, Arabidopsis thaliana
atggcggatactgctagaggaacccatcacgatatcatcggcagagaccagtacccgatgatgggccgagaccgagaccagtaccagatgtccggacgaggatctgactactccaagtctaggcagattgctaaagctgcaactgctgtcacagctggtggttccctccttgttctctccagccttacccttgttggaactgtcatagctttgactgttgcaacacctctgctcgttatcttcagcccaatccttgtcccggctctcatcacagttgcactcctcatcaccggttttctttcctctggagggtttggcattgccgctataaccgttttctcttggatttacaagtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggtcaacacactact

SEQ ID NO: 2 - protein, 173 aa, Arabidopsis thaliana
MADTARGTHHDIIGRDQYPMMGRDRDQYQMSGRGSDYSKSRQIAKAATAVTAGGSLLVLSSLTLVGTVIALTVATPLLVIFSPILVPALITVALLITGFLSSGGFGIAAITVFSWIYKYATGEHPQGSDKLDSARMKLGSKAQDLKDRAQYYGQQHTGGEHDRDRTRGGQHTT

SEQ ID NO: 3 - DNA, 465 bp, Artificial Sequence
  Oleo-H23P open reading frame
atggcggatactgctagaggaacccatcacgatatcatcggcagagaccagtacccgatgatgggccgagaccgagaccagtaccagatgtccggacgaggatctgactactccaagtctagacagattgctaaaggcgcccttgttctctccagccttacccttgttggaactgtcatagctttgactgttgcaacacctctgctcgttatcttcagcccaatccttgtcccggctctcatcacagttgcactcctcatcaccggttttctttcctctggagggtttggcatttttaagtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggtcaacacactact

SEQ ID NO: 4 - protein, 155 aa, Artificial Sequence
  Oleo H23P protein
MADTARGTHHDIIGRDQYPMMGRDRDQYQMSGRGSDYSKSRQIAKGALVLSSLTLVGTVIALTVATPLLVIFSPILVPALITVALLITGFLSSGGFGIFKYATGEHPQGSDKLDSARMKLGSKAQDLKDRAQYYGQQHTGGEHDRDRTRGGQHTT

SEQ ID NO: 5 - DNA, 414 bp, Artificial Sequence
  Oleo-H3P open reading frame
atggcggatactgctagaggaacccatcacgatatcatcggcagagaccagtacccgatgatgggccgagaccgagaccagtaccagatgtccggacgaggatctgactactccaagtctagacagattgctaaaggcgccggaactgtcatagctttgactgttgcaacacctctgctcgttatcttcagcccaatccttgtcccggctctcatcacagttgcactcctcatcaccggtatttttaagtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggtcaacacactact

SEQ ID NO: 6 - protein, 138 aa, Artificial Sequence
  Oleo-H3P protein
MADTARGTHHDIIGRDQYPMMGRDRDQYQMSGRGSDYSKSRQIAKGAGTVIALTVATPLLVIFSPILVPALITVALLITGIFKYATGEHPQGSDKLDSARMKLGSKAQDLKDRAQYYGQQHTGGEHDRDRTRGGQHTT

SEQ ID NO: 7 - DNA, 351 bp, Artificial Sequence
  Oleo-P open reading frame
atggcggatactgctagaggaacccatcacgatatcatcggcagagaccagtacccgatgatgggccgagaccgagaccagtaccagatgtccggacgaggatctgactactccaagtctagacagattgctaaaggcgcccctctgctcgttatcttcagccccatccttgtcccgatttttaagtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggtcaacacactact

SEQ ID NO: 8 - protein, 117 aa, Artificial Sequence
  Oleo-P protein
MADTARGTHHDIIGRDQYPMMGRDRDQYQMSGRGSDYSKSRQIAKGAPLLVIFSPILVPIFKYATGEHPQGSDKLDSARMKLGSKAQDLKDRAQYYGQQHTGGEHDRDRTRGGQHTT

SEQ ID NO: 9 - DNA, 468 bp, Artificial Sequence
  Oleo-H12P open reading frame
atggcggatactgctagaggaacccatcacgatatcatcggcagagaccagtacccgatgatgggccgagaccgagaccagtaccagatgtccggacgaggatctgactactccaagtctaggcagattgctaaagctgcaactgctgtcacagctggtggttccctccttgttctctccagccttacccttgttgcgactcctcttctcgttatcttcagcccaatccttgtcccggccctatttctttcctctggagggtttggcattgccgctataaccgttttctcttggatttacaagtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggtcaacacactact

SEQ ID NO: 10 - protein, 156 aa, Artificial Sequence
  Oleo-H12P protein
MADTARGTHHDIIGRDQYPMMGRDRDQYQMSGRGSDYSKSRQIAKAATAVTAGGSLLVLSSLTLVATPLLVIFSPILVPALFLSSGGFGIAAITVFSWIYKYATGEHPQGSDKLDSARMKLGSKAQDLKDRAQYYGQQHTGGEHDRDRTRGGQHTT

SEQ ID NO: 11 - DNA, 417 bp, Artificial Sequence
  Oleo-H1P open reading frame
atggcggatactgctagaggaacccatcacgatatcatcggcagagaccagtacccgatgatgggccgagaccgagaccagtaccagatgtccggacgaggatctgactactccaagtctaggcagattgctaaagctgcaactgctgtcacagctggtggttccctcgcgactcctcttctcgttatcttcagcccaatccttgtcccggccctaattgcagctataaccgttttctcttggatttacaagtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggtcaacacactact

SEQ ID NO: 12 - protein, 139 aa, Artificial Sequence
  Oleo-H1P protein
MADTARGTHHDIIGRDQYPMMGRDRDQYQMSGRGSDYSKSRQIAKAATAVTAGGSLATPLLVIFSPILVPALIAAITVFSWIYKYATGEHPQGSDKLDSARMKLGSKAQDLKDRAQYYGQQHTGGEHDRDRTRGGQHTT

SEQ ID NO: 13 - DNA, 690 bp, Artificial Sequence
  Oleo-double open reading frame
atggcggatactgctagaggaacccatcacgatatcatcggcagagaccagtacccgatgatgggccgagaccgagaccagtaccagatgtccggacgaggatctgactactccaagtctagacagattgctaaaggcgccactgctgtcactgctggtggttccctccttgttctctccagccttacccttgttggaactgtgatagctttgactgttgtgactcctgcggtcacagctagtggttccctccttgttctctccagccttacccttgttggaactgtcatagctttgactgttgcaacacctctgctcgttatcttcagcccaatccttgtcccggctctcatcacagttgcactcctcatcaccggttttctttcctctggagggtttggcattgccgctataagtgttttctcttgggcggccctcatcacagttgcactcctcatcaccggttttctttcctctggagggtttggcattgccgctataagcgttttctcttggatttttaagtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggtcaacacactact

SEQ ID NO: 14 - protein, 230 aa, Artificial Sequence
  Oleo-double protein
MADTARGTHHDIIGRDQYPMMGRDRDQYQMSGRGSDYSKSRQIAKGATAVTAGGSLLVLSSLTLVGTVIALTVVTPAVTASGSLLVLSSLTLVGTVIALTVATPLLVIFSPILVPALITVALLITGFLSSGGFGIAAISVFSWAALITVALLITGFLSSGGFGIAAISVFSWIFKYATGEHPQGSDKLDSARMKLGSKAQDLKDRAQYYGQQHTGGEHDRDRTRGGQHTT

SEQ ID NO: 15 - protein, 118 aa, Arabidopsis thaliana
MADTARGTHHDIIGRDQYPMMGRDRDQYQMSGRGSDYSKSRQIAKAATAVTAGGSLLVLSSLTLVGTVIALTVATPLLVIFSPILVPALITVALLITGFLSSGGFGIAAITVFSWIYK

SEQ ID NO: 16 - protein, 187 aa, Brassica napus
MADTARTHHDVTSRDQYPRDRDQYSMIGRDRDQYSMMGRDRDQYNMYGRDYSKSRQIAKAVTAVTAGGSLLVLSSLTLVGTVIALTVATPLLVIFSPILVPALITVALLITGFLSSGGFAIAAITVFSWIYKYATGEHPQGSDKLDSARMKLGTKAQDIKDRAQYYGQQHTGGEHDRDRTRGGQHTT

SEQ ID NO: 17 - DNA, 141 bp, Artificial Sequence
  N-terminal domain from Oleo-FL
atggcggatactgctagaggaacccatcacgatatcatcggcagagaccagtacccgatgatgggccgagaccgagaccagtaccagatgtccggacgaggatctgactactccaagtctaggcagattgctaaagctgca

SEQ ID NO: 18 - DNA, 141 bp, Artificial Sequence
  N-terminal domain from Oleo-FL
atggcggatactgctagaggaacccatcacgatatcatcggcagagaccagtacccgatgatgggccgagaccgagaccagtaccagatgtccggacgaggatctgactactccaagtctagacagattgctaaaggcgcc

SEQ ID NO: 19 - DNA, 27 bp, Artificial Sequence
  Hydrophobic HN Domain 1a from Oleo-FL
actgctgtcacagctggtggttccctc

SEQ ID NO: 20 - DNA, 24 bp, Artificial Sequence
  Hydrophobic HN Domain 1a from Oleo-FL
gcggtcacagctagtggttccctc

SEQ ID NO: 21 - DNA, 27 bp, Artificial Sequence
  Hydrophobic HN Domain 2a from Oleo-FL
cttgttctctccagccttacccttgtt

SEQ ID NO: 22 - DNA, 24 bp, Artificial Sequence
  Hydrophobic HN Domain 3a from Oleo-FL
ggaactgtcatagctttgactgtt

SEQ ID NO: 23 - DNA, 36 bp, Artificial Sequence
  Proline Knot from Oleo-FL
cctctgctcgttatcttcagcccaatccttgtcccg

SEQ ID NO: 24 - DNA, 48 bp, Artificial Sequence
  Proline Knot from Oleo-FL
gcaacacctctgctcgttatcttcagcccaatccttgtcccggctctc

SEQ ID NO: 25 - DNA, 27 bp, Artificial Sequence
  Hydrophobic HN Domain 1b from Oleo-FL
attgccgctataaccgttttctcttgg

SEQ ID NO: 26 - DNA, 27 bp, Artificial Sequence
  Hydrophobic HN Domain 1b from Oleo-FL
attgccgctataagcgttttctcttgg

SEQ ID NO: 27 - DNA, 24 bp, Artificial Sequence
  Hydrophobic HN Domain 2b from Oleo-FL
tttctttcctctggagggtttggc

SEQ ID NO: 28 - DNA, 27 bp, Artificial Sequence
  Hydrophobic HN Domain 3b form Oleo FL
atcacagttgcactcctcatcaccggt

SEQ ID NO: 29 - DNA, 174 bp, Artificial Sequence
  C-terminal domain from Oleo-FL
atttacaagtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggtcaacacactact

SEQ ID NO: 30 - DNA, 174 bp, Artificial Sequence
  C-terminal domain from Oleo-FL
atttttaagtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggtcaacacactact

SEQ ID NO: 31 - protein, 47 aa, Artificial Sequence
  N-terminal domain from Oleo-FL
MADTARGTHHDIIGRDQYPMMGRDRDQYQMSGRGSDYSKSRQIAKAA

SEQ ID NO: 32 - protein, 94 aa, Artificial Sequence
  N-terminal domain from Oleo-FL
MADTARGTHHDIIGRDQYPMMGRDRDQYQMSGRGSDYSKSRQIAKAAMADTARGTHHDIIGRDQYPMMGRDRDQYQMSGRGSDYSKSRQIAKGA

SEQ ID NO: 33 - protein, 9 aa, Artificial Sequence
  Hydrophobic HN Domain 1a from Oleo-FL
TAVTAGGSL

SEQ ID NO: 34 - protein, 9 aa, Artificial Sequence
  Hydrophobic HN Domain 1a from Oleo-FL
TAVTASGSL

SEQ ID NO: 35 - protein, 9 aa, Artificial Sequence
  Hydrophobic HN Domain 2a from Oleo-FL
LVLSSLTLV

SEQ ID NO: 36 - protein, 8 aa, Artificial Sequence
  Hydrophobic HN Domain 3a from Oleo-FL
GTVIALTV

SEQ ID NO: 37 - protein, 12 aa, Artificial Sequence
  Proline Knot from Oleo-FL
PLLVIFSPILVP

SEQ ID NO: 38 - protein, 16 aa, Artificial Sequence
  Proline Knot from Oleo-FL
ATPLLVIFSPILVPAL

SEQ ID NO: 39 - protein, 9 aa, Artificial Sequence
  Hydrophobic HC Domain 1b from Oleo-FL
IAAITVFSW

SEQ ID NO: 40 - protein, 9 aa, Artificial Sequence
  Hydrophobic HC Domain 1b from Oleo-FL
IAAISVFSW

SEQ ID NO: 41 - protein, 8 aa, Artificial Sequence
  Hydrophobic HC Domain 2b from Oleo-FL
FLSSGGFG

SEQ ID NO: 42 - protein, 9 aa, Artificial Sequence
  Hydrophobic HC Domain 3b from Oleo-FL
ITVALLITG

SEQ ID NO: 43 - protein, 58 aa, Artificial Sequence
  C-terminal domain from Oleo-FL
IYKYATGEHPQGSDKLDSARMKLGSKAQDLKDRAQYYGQQHTGGEHDRDRTRGGQHTT

SEQ ID NO: 44 - protein, 58 aa, Artificial Sequence
  C-terminal domain from Oleo-FL
IFKYATGEHPQGSDKLDSARMKLGSKAQDLKDRAQYYGQQHTGGEHDRDRTRGGQHTT

SEQ ID NO: 45 - DNA, 32 bp, Artificial Sequence
  forward primer NTD2
tattctcgagccatggcggatactgctagagg

SEQ ID NO: 46 - DNA, 29 bp, Artificial Sequence
  Reverse primer NTR
cagtggcgcctttagcaatctgtctagac

SEQ ID NO: 47 - DNA, 29 bp, Artificial Sequence
  Forward primer HN2D
cagctggtggtggcgcccttgttctctcc

SEQ ID NO: 48 - DNA, 27 bp, Artificial Sequence
  Reverse primer HC2R
ttatattaaaaatgccaaaccctccag

SEQ ID NO: 49 - DNA, 28 bp, Artificial Sequence
  Forward primer CTD
tggatttttaagtacgcaacgggagagc

SEQ ID NO: 50 - DNA, 32 bp, Artificial Sequence
  Reverse primer CTR
agccatactagtagtgtgttgaccaccacgag

SEQ ID NO: 51 - DNA, 31 bp, Artificial Sequence
  Forward primer HN3D
ccttagcggcgccggaactgtcatagctttg

SEQ ID NO: 52 - DNA, 28 bp, Artificial Sequence
  Reverse primer HC3R
agagttaaaaataccggtgatgaggagt

SEQ ID NO: 53 - DNA, 30 bp, Artificial Sequence
  Forward primer PKMD
actgttggcgcccctctgctcgttatcttc

SEQ ID NO: 54 - DNA, 29 bp, Artificial Sequence
  Reverse primer PKMR
tgtgttaaaaatcgggacaaggatggggc

SEQ ID NO: 55 - DNA, 32 bp, Artificial Sequence
  Reverse primer HN2R
ataggagtcgcaacaagggtaaggctggagag

SEQ ID NO: 56 - DNA, 32 bp, Artificial Sequence
  Forward primer PKMD-Hinf
ttgtgactcctcttctcgttatcttcagccca

SEQ ID NO: 57 - DNA, 34 bp, Artificial Sequence
  Reverse primer PKMR-Sau961
atgagggccgggacaaggattggactgaagataa

SEQ ID NO: 58 - DNA, 28 bp, Artificial Sequence
  Forward primer HC2D
tcatggccctatttctttcctctggagg

SEQ ID NO: 59 - DNA, 30 bp, Artificial Sequence
  Reverse primer HN1R
ataggagtcgcgagggaaccaccagctgtg

SEQ ID NO: 60 - DNA, 32 bp, Artificial Sequence
  Forward primer HC1D
gagaggccctaattgcagctataaccgttttc

SEQ ID NO: 61 - DNA, 27 bp, Artificial Sequence
  Forward primer HN1D
ctaaaggcgccactgctgtcactgctg

SEQ ID NO: 62 - DNA, 31 bp, Artificial Sequence
  Reverse primer  DoubleNR
agaggagtcacaacagtcaaagctatcacag

SEQ ID NO: 63 - DNA, 31 bp, Artificial Sequence
  Forward primer DoubleND
tacagggccatccaagagaaaacacttatag

SEQ ID NO: 64 - DNA, 31 bp, Artificial Sequence; identical to SEQ ID NO: 63
  Reverse primer DoubleCR

SEQ ID NO: 65 - DNA, 29 bp, Artificial Sequence
  Forward primer DoubleCD
tcccggccctcatcacagttgcactcctc

SEQ ID NO: 66 - DNA, 28 bp, Artificial Sequence
  Reverse primer HC1R
gcgtagttaaaaatccaagagaaaacgg

SEQ ID NO: 67 - DNA, 34 bp, Artificial Sequence
  Forward primer GFPder
ttactactagtatggcatctaaaggagaagaact

SEQ ID NO: 68 - DNA, 40 bp, Artificial Sequence
  Reverse primer GFPrev
atcggagctcctgcagttatttgtatagttcatccatgcc